SEQUENCE LISTING (32 sequences)

SEQ ID NO: 1 - DNA, 541 bp, synthetic construct
  source 1..541: mol_type = other DNA
  CDS 20..535: protein_id = 2; translation = MYRKLAVISAFLATARAMGLSDGEWQLVLNAWGKVEADVAGHGQ EVLIRLFTGHPETLEKFDKFKHLKTEAEMKASEDLKKHGNTVLTALGGILKKKGHHEA EVKHLAESHANKHKIPVKYLEFISDAIIHVLHAKHPSDFGADAQAAMSKALELFRNDM AAQYKVLGFHG
  sig_peptide 20..70: note = Trichoderma ressei CBH1 signal peptide
  misc_feature 1..8: note = PacI site
  misc_feature 536..541: note = XbaI site
  misc_feature 9..19: note = Kozak sequence
ttaattaaccaacatgagaatgtatcggaagttggccgtcatctcggccttcttggccacagctcgtgctatgggattgtcagacggtgagtggcagctggtgctcaacgcttggggcaaggtcgaggccgacgttgccggtcatggccaggaggtgcttattcgcctattcaccgggcaccccgagacgctggagaagtttgacaagttcaagcacctcaagacagaagccgagatgaaggcgagcgaagatttgaaaaaacacggcaacaccgttctcacggcgcttgggggcatcctcaagaagaagggccaccacgaggcagaagtcaaacatctcgcagagagccatgccaacaagcacaagattccggtcaagtatctggagttcatctctgatgccatcatccacgtcctgcacgccaagcatccttcggactttggcgccgatgctcaggctgcgatgtccaaggccctcgaactgtttcggaatgacatggcggcacaatacaaggtgctgggcttccacggatgatctaga

SEQ ID NO: 2 - protein, 171 aa, synthetic construct
MYRKLAVISAFLATARAMGLSDGEWQLVLNAWGKVEADVAGHGQEVLIRLFTGHPETLEKFDKFKHLKTEAEMKASEDLKKHGNTVLTALGGILKKKGHHEAEVKHLAESHANKHKIPVKYLEFISDAIIHVLHAKHPSDFGADAQAAMSKALELFRNDMAAQYKVLGFHG

SEQ ID NO: 3 - DNA, 1895 bp, synthetic construct
  source 1..1895: mol_type = other DNA
  CDS 12..1895: protein_id = 4; translation = MDSVLRQSKAMCPFLKSASPATLRAMSTAARPKASLPAAGAAAA SPCGGTMSKLQLYAHRCPVMGKAMAVQSSKYGAAAGIRAFSAHSKTGKARLHTSRNQE ARAVESPLLEGRKAAPPPPPGVTTAAARKDAPPVSAVPSPFSASGKFSYEDFYESQLE KKRQDKSYRYFNNINRLAKEFPVAHMAKNEDKVTVWCANDYLGMGGNQHVLNTMHEAL EKYGAGAGGTRNISGHNKHAVELEATLAKLHATESALVFSSCYVANDATLATLGSKLP DCVILSDSLNHASMIQGIRHSGTKKIVFKHNDVADLEAKLASLPLHVPKIIAFESVYS MCGSIGPIEEICNLAEKYGALTFLDEVHAVGMYGPTGAGVAEHLDWEAHASGKPRGTI MDRIDIFTGTLAKAYGCVGGYIAGSAKFVDMIRSLAPGFIFTSTLPPATMAGAKASIE YQMAYDGDRRLQQLYTRAVKEELNARDIPVIPNPSHIVPILVGNAALAKEASDLLLHD YKIYVQAINYPTVPIGQERLRVTPTPGHTKELREKLVTALDEIWTRLDIKRTSQWAAE GGFIGVGVEGNVPEPLWTDKLLGIEQAAKEVKAAGVPENGITEALLAREGLNNSRVMD VSA
  misc_feature 1..11: note = Kozak sequence
ccaacatgagaatggactccgtcctgcggcagtccaaggccatgtgccccttcctgaagtccgcctcccccgccaccctgcgggccatgtccaccgccgcccggcccaaggcctccctgcccgccgccggcgccgccgccgcctccccctgcggcggcaccatgtccaagctgcagctgtatgcccaccggtgccccgtcatgggcaaggccatggccgtccagtcctccaagtatggcgccgccgccggcatccgggccttctccgcccactccaagaccggcaaggcccggctgcacacctcccggaaccaggaggcccgggccgtcgagtcccccctgctggagggccggaaggccgcccccccccccccccccggcgtcaccaccgccgccgcccggaaggacgccccccccgtctccgccgtcccctcccccttctccgcctccggcaagttctcctatgaggacttctatgagtcccagctggagaagaagcggcaggacaagtcctatcggtatttcaacaacatcaaccggctggccaaggagttccccgtcgcccacatggccaagaacgaggacaaggtcaccgtctggtgcgccaacgactatctgggcatgggcggcaaccagcacgtcctgaacaccatgcacgaggccctggagaagtatggcgccggcgccggcggcacccggaacatctccggccacaacaagcacgccgtcgagctggaggccaccctggccaagctgcacgccaccgagtccgccctggtcttctcctcctgctatgtcgccaacgacgccaccctggccaccctgggctccaagctgcccgactgcgtcatcctgtccgactccctgaaccacgcctccatgatccagggcatccggcactccggcaccaagaagatcgtcttcaagcacaacgacgtcgccgacctggaggccaagctggcctccctgcccctgcacgtccccaagatcatcgccttcgagtccgtctattccatgtgcggctccatcggccccatcgaggagatctgcaacctggccgagaagtatggcgccctgaccttcctggacgaggtccacgccgtcggcatgtatggccccaccggcgccggcgtcgccgagcacctggactgggaggcccacgcctccggcaagccccggggcaccatcatggaccggatcgacatcttcaccggcaccctggccaaggcctatggctgcgtcggcggctatatcgccggctccgccaagttcgtcgacatgatccggtccctggcccccggcttcatcttcacctccaccctgccccccgccaccatggccggcgccaaggcctccatcgagtatcagatggcctatgacggcgaccggcggctgcagcagctgtatacccgggccgtcaaggaggagctgaacgcccgggacatccccgtcatccccaacccctcccacatcgtccccatcctggtcggcaacgccgccctggccaaggaggcctccgacctgctgctgcacgactataagatctatgtccaggccatcaactatcccaccgtccccatcggccaggagcggctgcgggtcacccccacccccggccacaccaaggagctgcgggagaagctggtcaccgccctggacgagatctggacccggctggacatcaagcggacctcccagtgggccgccgagggcggcttcatcggcgtcggcgtcgagggcaacgtccccgagcccctgtggaccgacaagctgctgggcatcgagcaggccgccaaggaggtcaaggccgccggcgtccccgagaacggcatcaccgaggccctgctggcccgggagggcctgaacaactcccgggtcatggacgtctccgcctaa

SEQ ID NO: 4 - protein, 627 aa, synthetic construct
MDSVLRQSKAMCPFLKSASPATLRAMSTAARPKASLPAAGAAAASPCGGTMSKLQLYAHRCPVMGKAMAVQSSKYGAAAGIRAFSAHSKTGKARLHTSRNQEARAVESPLLEGRKAAPPPPPGVTTAAARKDAPPVSAVPSPFSASGKFSYEDFYESQLEKKRQDKSYRYFNNINRLAKEFPVAHMAKNEDKVTVWCANDYLGMGGNQHVLNTMHEALEKYGAGAGGTRNISGHNKHAVELEATLAKLHATESALVFSSCYVANDATLATLGSKLPDCVILSDSLNHASMIQGIRHSGTKKIVFKHNDVADLEAKLASLPLHVPKIIAFESVYSMCGSIGPIEEICNLAEKYGALTFLDEVHAVGMYGPTGAGVAEHLDWEAHASGKPRGTIMDRIDIFTGTLAKAYGCVGGYIAGSAKFVDMIRSLAPGFIFTSTLPPATMAGAKASIEYQMAYDGDRRLQQLYTRAVKEELNARDIPVIPNPSHIVPILVGNAALAKEASDLLLHDYKIYVQAINYPTVPIGQERLRVTPTPGHTKELREKLVTALDEIWTRLDIKRTSQWAAEGGFIGVGVEGNVPEPLWTDKLLGIEQAAKEVKAAGVPENGITEALLAREGLNNSRVMDVSA

SEQ ID NO: 5 - DNA, 1172 bp, synthetic construct
  source 1..1172: mol_type = other DNA
  CDS 12..1172: protein_id = 6; translation = MSFSSLVQELSLRDANNARRPGPAPSVSTSTFDDRASHISRTMS GYASTAATSVSISGDIGSQLHGGYFHPLARSWQSERQLTKSMLIYPIFVTDGEDDMIH VASLPGQFQISIDRLVGFLEPLVHKGLRSVMLFGVPQKPGTKDALGSAADDPEGPVIQ AIQLIRRRFPQIFICADVCLCEYTSHGHCGILRDDSSLNNQMSVDRISDVAIAYAKAG AHCVAPSDMNDGRIRAIKLKLIEEGIAHKTLLMSYSAKFSGCLYGPFRDAAGSAPAFG DRRCYQLPPGGRGLARRAIVRDINEGADIIMVKPAGQYLDIISDAKDLGKDMPVAAYQ VSGEYSMIHAAAKAGVFDLKTMAFESTESILRAGATIVISYFTPQFLDWLEN
  misc_feature 1..11: note = Kozak sequence
ccaacatgagaatgagcttcagcagcctcgtccaggagctcagcctccgcgacgccaacaacgcccgccgccccggccccgcccccagcgtcagcaccagcaccttcgacgaccgcgccagccacatcagccgcaccatgagcggctacgccagcaccgccgccaccagcgtcagcatcagcggcgacatcggcagccagctccacggcggctacttccaccccctcgcccgcagctggcagagcgagcgccagctcaccaagagcatgctcatctaccccatcttcgtcaccgacggcgaggacgacatgatccacgtcgccagcctccccggccagttccagatcagcatcgaccgcctcgtcggcttcctcgagcccctcgtccacaagggcctccgcagcgtcatgctcttcggcgtcccccagaagcccggcaccaaggacgccctcggcagcgccgccgacgaccccgagggccccgtcatccaggccatccagctcatccgccgccgcttcccccagatcttcatctgcgccgacgtctgcctctgcgagtacaccagccacggccactgcggcatcctccgcgacgacagcagcctcaacaaccagatgagcgtcgaccgcatcagcgacgtcgccatcgcctacgccaaggccggcgcccactgcgtcgcccccagcgacatgaacgacggccgcatccgcgccatcaagctcaagctcatcgaggagggcatcgcccacaagaccctcctcatgagctacagcgccaagttcagcggctgcctctacggccccttccgcgacgccgccggcagcgcccccgccttcggcgaccgccgctgctaccagctcccccccggcggccgcggcctcgcccgccgcgccatcgtccgcgacatcaacgagggcgccgacatcatcatggtcaagcccgccggccagtacctcgacatcatcagcgacgccaaggacctcggcaaggacatgcccgtcgccgcctaccaggtcagcggcgagtacagcatgatccacgccgccgccaaggccggcgtcttcgacctcaagaccatggccttcgagagcaccgagagcatcctccgcgccggcgccaccatcgtcatcagctacttcaccccccagttcctcgactggctcgagaactaa

SEQ ID NO: 6 - protein, 386 aa, synthetic construct
MSFSSLVQELSLRDANNARRPGPAPSVSTSTFDDRASHISRTMSGYASTAATSVSISGDIGSQLHGGYFHPLARSWQSERQLTKSMLIYPIFVTDGEDDMIHVASLPGQFQISIDRLVGFLEPLVHKGLRSVMLFGVPQKPGTKDALGSAADDPEGPVIQAIQLIRRRFPQIFICADVCLCEYTSHGHCGILRDDSSLNNQMSVDRISDVAIAYAKAGAHCVAPSDMNDGRIRAIKLKLIEEGIAHKTLLMSYSAKFSGCLYGPFRDAAGSAPAFGDRRCYQLPPGGRGLARRAIVRDINEGADIIMVKPAGQYLDIISDAKDLGKDMPVAAYQVSGEYSMIHAAAKAGVFDLKTMAFESTESILRAGATIVISYFTPQFLDWLEN

SEQ ID NO: 7 - DNA, 1013 bp, synthetic construct
  source 1..1013: mol_type = other DNA
  CDS 12..1013: protein_id = 8; translation = MSEPPRAPLPPLRIGTRRSNLAVVQAEGIRDSLQRIAPDRAFEI VALHTLGDKDKSTELYNFGAKNLWTAELEDKLTSRDLDVIVHCLKDMPTRLPESCDLA AIPLRDDPRDALIVKAGMPYTTLKTLPEGAIVGTSSVRRSAQLRRLYPHLRFANLRGN VETRLAKVDDPESEYTCMIMSAAGLERVGLKHRINQYLGSKDGGILHAVGQGALGLEI RKGDDAVLELLGRLADEKSTLACLAERALLYTLEGGCTVPIGVETEWVGDGARVLRMR AIVVSLDGTRSVEDSLDAEVRTNDEARAFGKELAERLVKAGADAILKEIVANRPPKN
  misc_feature 1..11: note = Kozak sequence
ccaacatgagaatgagcgagcccccccgcgcccccctcccccccctccgcatcggcacccgccgcagcaacctcgccgtcgtccaggccgagggcatccgcgacagcctccagcgcatcgcccccgaccgcgccttcgagatcgtcgccctccacaccctcggcgacaaggacaagagcaccgagctctacaacttcggcgccaagaacctctggaccgccgagctcgaggacaagctcaccagccgcgacctcgacgtcatcgtccactgcctcaaggacatgcccacccgcctccccgagagctgcgacctcgccgccatccccctccgcgacgacccccgcgacgccctcatcgtcaaggccggcatgccctacaccaccctcaagaccctccccgagggcgccatcgtcggcaccagcagcgtccgccgcagcgcccagctccgccgcctctacccccacctccgcttcgccaacctccgcggcaacgtcgagacccgcctcgccaaggtcgacgaccccgagagcgagtacacctgcatgatcatgagcgccgccggcctcgagcgcgtcggcctcaagcaccgcatcaaccagtacctcggcagcaaggacggcggcatcctccacgccgtcggccagggcgccctcggcctcgagatccgcaagggcgacgacgccgtcctcgagctcctcggccgcctcgccgacgagaagagcaccctcgcctgcctcgccgagcgcgccctcctctacaccctcgagggcggctgcaccgtccccatcggcgtcgagaccgagtgggtcggcgacggcgcccgcgtcctccgcatgcgcgccatcgtcgtcagcctcgacggcacccgcagcgtcgaggacagcctcgacgccgaggtccgcaccaacgacgaggcccgcgccttcggcaaggagctcgccgagcgcctcgtcaaggccggcgccgacgccatcctcaaggagatcgtcgccaaccgcccccccaagaactaa

SEQ ID NO: 8 - protein, 333 aa, synthetic construct
MSEPPRAPLPPLRIGTRRSNLAVVQAEGIRDSLQRIAPDRAFEIVALHTLGDKDKSTELYNFGAKNLWTAELEDKLTSRDLDVIVHCLKDMPTRLPESCDLAAIPLRDDPRDALIVKAGMPYTTLKTLPEGAIVGTSSVRRSAQLRRLYPHLRFANLRGNVETRLAKVDDPESEYTCMIMSAAGLERVGLKHRINQYLGSKDGGILHAVGQGALGLEIRKGDDAVLELLGRLADEKSTLACLAERALLYTLEGGCTVPIGVETEWVGDGARVLRMRAIVVSLDGTRSVEDSLDAEVRTNDEARAFGKELAERLVKAGADAILKEIVANRPPKN

SEQ ID NO: 9 - DNA, 1016 bp, synthetic construct
  source 1..1016: mol_type = other DNA
  CDS 12..1016: protein_id = 10; translation = MSSPTSSDQQPIPVLLLKTKSSPTDAYEDLFSSQSTSKPSFAPT FVPVLQHRFEDTGVNRLRDLLRRKRIGTSPDCDFGGLIFTSQRAVEAFSHVVREDEAS KDPSWPHLQHIPIYSVGPATTRALSAVPQDPPLQIFGSQTGNGDALAHFILDHYAQCK QHDDKNNQLLPPLLFLVGEQRRDIIPRTLTDPSLPAHRRIPVTEEVVYGTGEMPSFPE DFSSVLNNNNNNNETRSSSSERWVVVFSPTGCDSMLRGLGLLDPETGNFAAAGGTTTS STTIRDGRTFVATIGPTTRNYLVHKFGFEPDVCAETPTPEGVLEGILAFRKRRLEKTT
  misc_feature 1..11: note = Kozak sequence
ccaacatgagaatgagcagccccaccagcagcgaccagcagcccatccccgtcctcctcctcaagaccaagagcagccccaccgacgcctacgaggacctcttcagcagccagagcaccagcaagcccagcttcgcccccaccttcgtccccgtcctccagcaccgcttcgaggacaccggcgtcaaccgcctccgcgacctcctccgccgcaagcgcatcggcaccagccccgactgcgacttcggcggcctcatcttcaccagccagcgcgccgtcgaggccttcagccacgtcgtccgcgaggacgaggccagcaaggaccccagctggccccacctccagcacatccccatctacagcgtcggccccgccaccacccgcgccctcagcgccgtcccccaggacccccccctccagatcttcggcagccagaccggcaacggcgacgccctcgcccacttcatcctcgaccactacgcccagtgcaagcagcacgacgacaagaacaaccagctcctcccccccctcctcttcctcgtcggcgagcagcgccgcgacatcatcccccgcaccctcaccgaccccagcctccccgcccaccgccgcatccccgtcaccgaggaggtcgtctacggcaccggcgagatgcccagcttccccgaggacttcagcagcgtcctcaacaacaacaacaacaacaacgagacccgcagcagcagcagcgagcgctgggtcgtcgtcttcagccccaccggctgcgacagcatgctccgcggcctcggcctcctcgaccccgagaccggcaacttcgccgccgccggcggcaccaccaccagcagcaccaccatccgcgacggccgcaccttcgtcgccaccatcggccccaccacccgcaactacctcgtccacaagttcggcttcgagcccgacgtctgcgccgagacccccacccccgagggcgtcctcgagggcatcctcgccttccgcaagcgccgcctcgagaagaccacctaa

SEQ ID NO: 10 - protein, 334 aa, synthetic construct
MSSPTSSDQQPIPVLLLKTKSSPTDAYEDLFSSQSTSKPSFAPTFVPVLQHRFEDTGVNRLRDLLRRKRIGTSPDCDFGGLIFTSQRAVEAFSHVVREDEASKDPSWPHLQHIPIYSVGPATTRALSAVPQDPPLQIFGSQTGNGDALAHFILDHYAQCKQHDDKNNQLLPPLLFLVGEQRRDIIPRTLTDPSLPAHRRIPVTEEVVYGTGEMPSFPEDFSSVLNNNNNNNETRSSSSERWVVVFSPTGCDSMLRGLGLLDPETGNFAAAGGTTTSSTTIRDGRTFVATIGPTTRNYLVHKFGFEPDVCAETPTPEGVLEGILAFRKRRLEKTT

SEQ ID NO: 11 - DNA, 1109 bp, synthetic construct
  source 1..1109: mol_type = other DNA
  CDS 12..1109: protein_id = 12; translation = MEHNFEPLKNDLLLRAAWGQTVERPPMWVMRQAGRYLPEYHEAK GTRDFFECCRDAEVASTLTLQPVERFAGLLDAAIIFSDILVIPQAMGMQVEMVDKKGP HFPSPLQDPEDGQYGRVLAGDVDVAAELGYVYEAITLTRRKLAGRVPLIGFCGAPWTL FCYMVEGGGTNLFAQSKKWIYKYPEESKKLLAKIADVCVEHLALQVKAGAQLVMVFDS WAGELGPASFKEFSEPYLAHIAQKLPQKLQELGLDKVPMTVFPKGAWYALDSACNLGY NVVGMDWLQDPAEAVRIRGDRNIVFQGNADPGVLYGSKAAITKAVEDMVKGFWVGNKG WIANLGHGITPGVNPDNLKFFFEEIHRLTKS
  misc_feature 1..11: note = Kozak sequence
ccaacatgagaatggagcacaacttcgagcccctcaagaacgacctcctcctccgcgccgcctggggccagaccgtcgagcgcccccccatgtgggtcatgcgccaggccggccgctacctccccgagtaccacgaggccaagggcacccgcgacttcttcgagtgctgccgcgacgccgaggtcgccagcaccctcaccctccagcccgtcgagcgcttcgccggcctcctcgacgccgccatcatcttcagcgacatcctcgtcatcccccaggccatgggcatgcaggtcgagatggtcgacaagaagggcccccacttccccagccccctccaggaccccgaggacggccagtacggccgcgtcctcgccggcgacgtcgacgtcgccgccgagctcggctacgtctacgaggccatcaccctcacccgccgcaagctcgccggccgcgtccccctcatcggcttctgcggcgccccctggaccctcttctgctacatggtcgagggcggcggcaccaacctcttcgcccagagcaagaagtggatctacaagtaccccgaggagagcaagaagctcctcgccaagatcgccgacgtctgcgtcgagcacctcgccctccaggtcaaggccggcgcccagctcgtcatggtcttcgacagctgggccggcgagctcggccccgccagcttcaaggagttcagcgagccctacctcgcccacatcgcccagaagctcccccagaagctccaggagctcggcctcgacaaggtccccatgaccgtcttccccaagggcgcctggtacgccctcgacagcgcctgcaacctcggctacaacgtcgtcggcatggactggctccaggaccccgccgaggccgtccgcatccgcggcgaccgcaacatcgtcttccagggcaacgccgaccccggcgtcctctacggcagcaaggccgccatcaccaaggccgtcgaggacatggtcaagggcttctgggtcggcaacaagggctggatcgccaacctcggccacggcatcacccccggcgtcaaccccgacaacctcaagttcttcttcgaggagatccaccgcctcaccaagagctaa

SEQ ID NO: 12 - protein, 365 aa, synthetic construct
MEHNFEPLKNDLLLRAAWGQTVERPPMWVMRQAGRYLPEYHEAKGTRDFFECCRDAEVASTLTLQPVERFAGLLDAAIIFSDILVIPQAMGMQVEMVDKKGPHFPSPLQDPEDGQYGRVLAGDVDVAAELGYVYEAITLTRRKLAGRVPLIGFCGAPWTLFCYMVEGGGTNLFAQSKKWIYKYPEESKKLLAKIADVCVEHLALQVKAGAQLVMVFDSWAGELGPASFKEFSEPYLAHIAQKLPQKLQELGLDKVPMTVFPKGAWYALDSACNLGYNVVGMDWLQDPAEAVRIRGDRNIVFQGNADPGVLYGSKAAITKAVEDMVKGFWVGNKGWIANLGHGITPGVNPDNLKFFFEEIHRLTKS

SEQ ID NO: 13 - DNA, 1253 bp, synthetic construct
  source 1..1253: mol_type = other DNA
  CDS 12..1253: protein_id = 14; translation = MAAPSAATRYVQRVASQVRLRSARAPRGQLTLSRWASTASGSRR GPASFGGSSSAAVWLAAAAIGVTAPLAYSLSTTEPAKLDVSSLAEKDEQKKREAAVTE ESPMRLRMEKFIKEQQQIIVKELERIDGKKFRKDEWTRPNGGGGITCVLQEGNVFEKA GVGVSVVYGSLPKPAIMKMRENHKNLDPDVESLEFFAAGLSMVLHPSNPMAPTVHLNY RYFETANPDGSSQAWWFGGGCDLTPSYLFDEDAIHFHKTIKEACDSHDKTYYPRFKKW CDKYFFNKHRGECRGVGGIFFDDLDETERDQENTFAFVQDCLKAFLPSYIPILEKRKD MPFDAKEKEWQQIRRGKYVEFNLVHDRGTAFGLNTPGSRVESILMSLPLTASWKYMYE PEPKSREQRLVDVLKEPKEWV
  misc_feature 1..11: note = Kozak sequence
ccaacatgagaatggccgcccccagcgccgccacccgctacgtccagcgcgtcgccagccaggtccgcctccgcagcgcccgcgccccccgcggccagctcaccctcagccgctgggccagcaccgccagcggcagccgccgcggccccgccagcttcggcggcagcagcagcgccgccgtctggctcgccgccgccgccatcggcgtcaccgcccccctcgcctacagcctcagcaccaccgagcccgccaagctcgacgtcagcagcctcgccgagaaggacgagcagaagaagcgcgaggccgccgtcaccgaggagagccccatgcgcctccgcatggagaagttcatcaaggagcagcagcagatcatcgtcaaggagctcgagcgcatcgacggcaagaagttccgcaaggacgagtggacccgccccaacggcggcggcggcatcacctgcgtcctccaggagggcaacgtcttcgagaaggccggcgtcggcgtcagcgtcgtctacggcagcctccccaagcccgccatcatgaagatgcgcgagaaccacaagaacctcgaccccgacgtcgagagcctcgagttcttcgccgccggcctcagcatggtcctccaccccagcaaccccatggcccccaccgtccacctcaactaccgctacttcgagaccgccaaccccgacggcagcagccaggcctggtggttcggcggcggctgcgacctcacccccagctacctcttcgacgaggacgccatccacttccacaagaccatcaaggaggcctgcgacagccacgacaagacctactacccccgcttcaagaagtggtgcgacaagtacttcttcaacaagcaccgcggcgagtgccgcggcgtcggcggcatcttcttcgacgacctcgacgagaccgagcgcgaccaggagaacaccttcgccttcgtccaggactgcctcaaggccttcctccccagctacatccccatcctcgagaagcgcaaggacatgcccttcgacgccaaggagaaggagtggcagcagatccgccgcggcaagtacgtcgagttcaacctcgtccacgaccgcggcaccgccttcggcctcaacacccccggcagccgcgtcgagagcatcctcatgagcctccccctcaccgccagctggaagtacatgtacgagcccgagcccaagagccgcgagcagcgcctcgtcgacgtcctcaaggagcccaaggagtgggtctaa

SEQ ID NO: 14 - protein, 413 aa, synthetic construct
MAAPSAATRYVQRVASQVRLRSARAPRGQLTLSRWASTASGSRRGPASFGGSSSAAVWLAAAAIGVTAPLAYSLSTTEPAKLDVSSLAEKDEQKKREAAVTEESPMRLRMEKFIKEQQQIIVKELERIDGKKFRKDEWTRPNGGGGITCVLQEGNVFEKAGVGVSVVYGSLPKPAIMKMRENHKNLDPDVESLEFFAAGLSMVLHPSNPMAPTVHLNYRYFETANPDGSSQAWWFGGGCDLTPSYLFDEDAIHFHKTIKEACDSHDKTYYPRFKKWCDKYFFNKHRGECRGVGGIFFDDLDETERDQENTFAFVQDCLKAFLPSYIPILEKRKDMPFDAKEKEWQQIRRGKYVEFNLVHDRGTAFGLNTPGSRVESILMSLPLTASWKYMYEPEPKSREQRLVDVLKEPKEWV

SEQ ID NO: 15 - DNA, 1712 bp, synthetic construct
  source 1..1712: mol_type = other DNA
  CDS 12..1712: protein_id = 16; translation = MSLKRADGAAMAFLRLSSAVCGRRRIPAGGLPADANVAVLGGGL TGLTTAYYLAKWLPPTAKITLLEASDRLGGWIKTDRVPVKVGGVEGVVSFERGPRSLS SLNKSTWRFDDLVLWDLALDLGLRVVSPPDKPRYIYYPDHLVPLPPHTSLAAFASEPL VLESLWAGFGYVLRRLFSRKAGVPVQDLSIADWIQHITGSRAVAENLASAMVHGIYGG DIYTLSARSVLDRFYWVHYLPALGPDVRHMALSEQVFMEAMGQDPLIRKLAQQPRGAL LNFGEAGMETLPIALADALNGQANVEVKLGAKVTGLEYESETEMMKITTTGQDDSPAE KYHKVISTLPSQHLARITDTLPSLASSHAVSIMTVNIWYPQTNLKPPGFGYLIPLSVP AEQNPERALGVFFDSDVGVRGPDEPAGTKLFVLMGGHYYDKKQPGASVRVPSEEEAIE QAKRVLERHLGIPQSTPCFAMARLASECIPQYVCGHQDTMAAADEDLRDSFDGRLAVA GGSYTKIGAMGALRNGYDIANTVVREDWLTTGLEQLEFPTQFCGVPTERIPVRRFSRR
  misc_feature 1..11: note = Kozak sequence
ccaacatgagaatgagcctcaagcgcgccgacggcgccgccatggccttcctccgcctcagcagcgccgtctgcggccgccgccgcatccccgccggcggcctccccgccgacgccaacgtcgccgtcctcggcggcggcctcaccggcctcaccaccgcctactacctcgccaagtggctcccccccaccgccaagatcaccctcctcgaggccagcgaccgcctcggcggctggatcaagaccgaccgcgtccccgtcaaggtcggcggcgtcgagggcgtcgtcagcttcgagcgcggcccccgcagcctcagcagcctcaacaagagcacctggcgcttcgacgacctcgtcctctgggacctcgccctcgacctcggcctccgcgtcgtcagcccccccgacaagccccgctacatctactaccccgaccacctcgtccccctccccccccacaccagcctcgccgccttcgccagcgagcccctcgtcctcgagagcctctgggccggcttcggctacgtcctccgccgcctcttcagccgcaaggccggcgtccccgtccaggacctcagcatcgccgactggatccagcacatcaccggcagccgcgccgtcgccgagaacctcgccagcgccatggtccacggcatctacggcggcgacatctacaccctcagcgcccgcagcgtcctcgaccgcttctactgggtccactacctccccgccctcggccccgacgtccgccacatggccctcagcgagcaggtcttcatggaggccatgggccaggaccccctcatccgcaagctcgcccagcagccccgcggcgccctcctcaacttcggcgaggccggcatggagaccctccccatcgccctcgccgacgccctcaacggccaggccaacgtcgaggtcaagctcggcgccaaggtcaccggcctcgagtacgagagcgagaccgagatgatgaagatcaccaccaccggccaggacgacagccccgccgagaagtaccacaaggtcatcagcaccctccccagccagcacctcgcccgcatcaccgacaccctccccagcctcgccagcagccacgccgtcagcatcatgaccgtcaacatctggtacccccagaccaacctcaagccccccggcttcggctacctcatccccctcagcgtccccgccgagcagaaccccgagcgcgccctcggcgtcttcttcgacagcgacgtcggcgtccgcggccccgacgagcccgccggcaccaagctcttcgtcctcatgggcggccactactacgacaagaagcagcccggcgccagcgtccgcgtccccagcgaggaggaggccatcgagcaggccaagcgcgtcctcgagcgccacctcggcatcccccagagcaccccctgcttcgccatggcccgcctcgccagcgagtgcatcccccagtacgtctgcggccaccaggacaccatggccgccgccgacgaggacctccgcgacagcttcgacggccgcctcgccgtcgccggcggcagctacaccaagatcggcgccatgggcgccctccgcaacggctacgacatcgccaacaccgtcgtccgcgaggactggctcaccaccggcctcgagcagctcgagttccccacccagttctgcggcgtccccaccgagcgcatccccgtccgccgcttcagccgccgctaa

SEQ ID NO: 16 - protein, 566 aa, synthetic construct
MSLKRADGAAMAFLRLSSAVCGRRRIPAGGLPADANVAVLGGGLTGLTTAYYLAKWLPPTAKITLLEASDRLGGWIKTDRVPVKVGGVEGVVSFERGPRSLSSLNKSTWRFDDLVLWDLALDLGLRVVSPPDKPRYIYYPDHLVPLPPHTSLAAFASEPLVLESLWAGFGYVLRRLFSRKAGVPVQDLSIADWIQHITGSRAVAENLASAMVHGIYGGDIYTLSARSVLDRFYWVHYLPALGPDVRHMALSEQVFMEAMGQDPLIRKLAQQPRGALLNFGEAGMETLPIALADALNGQANVEVKLGAKVTGLEYESETEMMKITTTGQDDSPAEKYHKVISTLPSQHLARITDTLPSLASSHAVSIMTVNIWYPQTNLKPPGFGYLIPLSVPAEQNPERALGVFFDSDVGVRGPDEPAGTKLFVLMGGHYYDKKQPGASVRVPSEEEAIEQAKRVLERHLGIPQSTPCFAMARLASECIPQYVCGHQDTMAAADEDLRDSFDGRLAVAGGSYTKIGAMGALRNGYDIANTVVREDWLTTGLEQLEFPTQFCGVPTERIPVRRFSRR

SEQ ID NO: 17 - DNA, 1277 bp, synthetic construct
  source 1..1277: mol_type = other DNA
  CDS 12..1277: protein_id = 18; translation = MAVRLTTQHFSRGLSRSAASPLALSQLASHARHLATPVHPVTQD ATGSKGPTAMVFLNMGGPSTLNEVGDFLSRLFADGDLIPLGRLQSYIGPLISARRTPK IQKQYDAIGGGSPIRKWSEYQSQEMCKILDKISPETAPHKPYVAFRYANPLTEEMYHK LLADGFGNGKGGRAVAFTQYPQYSCSTTGSSLNELWKWRQRLEGKTASETGNGTITWS VIDRWPTHPGLVEAFAQNIEAKLAEYPEERRRNAILLFSAHSLPMDVVNRGDPYPGEV AATVQAVMERLKFSNPYRLCWQSQVGPKAWLGPQTSDTVENYIAKGQKDLVLIPIAFT SDHIETLYELDIEVIGESGHADTVKRVDSLNGSPVFIKALADLAKSHLGSGQACSLQM GLRCPACKSERCAESKKFFASQQAALSSA
  misc_feature 1..11: note = Kozak sequence
ccaacatgagaatggccgtccgcctcaccacccagcacttcagccgcggcctcagccgcagcgccgccagccccctcgccctcagccagctcgccagccacgcccgccacctcgccacccccgtccaccccgtcacccaggacgccaccggcagcaagggccccaccgccatggtcttcctcaacatgggcggccccagcaccctcaacgaggtcggcgacttcctcagccgcctcttcgccgacggcgacctcatccccctcggccgcctccagagctacatcggccccctcatcagcgcccgccgcacccccaagatccagaagcagtacgacgccatcggcggcggcagccccatccgcaagtggagcgagtaccagagccaggagatgtgcaagatcctcgacaagatcagccccgagaccgccccccacaagccctacgtcgccttccgctacgccaaccccctcaccgaggagatgtaccacaagctcctcgccgacggcttcggcaacggcaagggcggccgcgccgtcgccttcacccagtacccccagtacagctgcagcaccaccggcagcagcctcaacgagctctggaagtggcgccagcgcctcgagggcaagaccgccagcgagaccggcaacggcaccatcacctggagcgtcatcgaccgctggcccacccaccccggcctcgtcgaggccttcgcccagaacatcgaggccaagctcgccgagtaccccgaggagcgccgccgcaacgccatcctcctcttcagcgcccacagcctccccatggacgtcgtcaaccgcggcgacccctaccccggcgaggtcgccgccaccgtccaggccgtcatggagcgcctcaagttcagcaacccctaccgcctctgctggcagagccaggtcggccccaaggcctggctcggcccccagaccagcgacaccgtcgagaactacatcgccaagggccagaaggacctcgtcctcatccccatcgccttcaccagcgaccacatcgagaccctctacgagctcgacatcgaggtcatcggcgagagcggccacgccgacaccgtcaagcgcgtcgacagcctcaacggcagccccgtcttcatcaaggccctcgccgacctcgccaagagccacctcggcagcggccaggcctgcagcctccagatgggcctccgctgccccgcctgcaagagcgagcgctgcgccgagagcaagaagttcttcgccagccagcaggccgccctcagcagcgcctaa

SEQ ID NO: 18 - protein, 421 aa, synthetic construct
MAVRLTTQHFSRGLSRSAASPLALSQLASHARHLATPVHPVTQDATGSKGPTAMVFLNMGGPSTLNEVGDFLSRLFADGDLIPLGRLQSYIGPLISARRTPKIQKQYDAIGGGSPIRKWSEYQSQEMCKILDKISPETAPHKPYVAFRYANPLTEEMYHKLLADGFGNGKGGRAVAFTQYPQYSCSTTGSSLNELWKWRQRLEGKTASETGNGTITWSVIDRWPTHPGLVEAFAQNIEAKLAEYPEERRRNAILLFSAHSLPMDVVNRGDPYPGEVAATVQAVMERLKFSNPYRLCWQSQVGPKAWLGPQTSDTVENYIAKGQKDLVLIPIAFTSDHIETLYELDIEVIGESGHADTVKRVDSLNGSPVFIKALADLAKSHLGSGQACSLQMGLRCPACKSERCAESKKFFASQQAALSSA

SEQ ID NO: 19 - DNA, 21 bp, synthetic construct
  source 1..21: mol_type = other DNA
atgtatcggaagttggccgtc

SEQ ID NO: 20 - DNA, 20 bp, synthetic construct
  source 1..20: mol_type = other DNA
gacctgcgacagacaaccaa

SEQ ID NO: 21 - DNA, 37 bp, synthetic construct
  source 1..37: mol_type = other DNA
gagacctgcaggatgaaaggagggagcattcttcgac

SEQ ID NO: 22 - DNA, 42 bp, synthetic construct
  source 1..42: mol_type = other DNA
cattctcatgttggttaattaagattgtgctgtagctgcgct

SEQ ID NO: 23 - DNA, 33 bp, synthetic construct
  source 1..33: mol_type = other DNA
gctcctgcagggacgcagaagaaggaaatcgcc

SEQ ID NO: 24 - DNA, 47 bp, synthetic construct
  source 1..47: mol_type = other DNA
cattctcatgttggttaattaatttgtatctgcgaattgagcttgcg

SEQ ID NO: 25 - DNA, 38 bp, synthetic construct
  source 1..38: mol_type = other DNA
ccacgccgatttcctgccaaaatggccaagttgaccag

SEQ ID NO: 26 - DNA, 41 bp, synthetic construct
  source 1..41: mol_type = other DNA
caggctttcgccacggagcttcagtcctgctcctcggccac

SEQ ID NO: 27 - DNA, 37 bp, synthetic construct
  source 1..37: mol_type = other DNA
ccgaggagcaggactgaagctccgtggcgaaagcctg

SEQ ID NO: 28 - DNA, 41 bp, synthetic construct
  source 1..41: mol_type = other DNA
gcactggtcaacttggccattttggcaggaaatcggcgtgg

SEQ ID NO: 29 - DNA, 37 bp, synthetic construct
  source 1..37: mol_type = other DNA
caaattaattaaccaacatgagaatgagaatggactc

SEQ ID NO: 30 - DNA, 28 bp, synthetic construct
  source 1..28: mol_type = other DNA
gcctctagattaggcggagacgtccatg

SEQ ID NO: 31 - DNA, 21 bp, synthetic construct
  source 1..21: mol_type = other DNA
ccaacatgagaatggactccg

SEQ ID NO: 32 - DNA, 23 bp, synthetic construct
  source 1..23: mol_type = other DNA
ttaggcggagacgtccatgaccc

(19) United States
(12) Patent Application Publication
(10) Pub. No.: US 2023/0340077 A1
(43) Pub. Date: Oct. 26, 2023

(54) PRODUCTION OF MYOGLOBIN FROM TRICHODERMA USING A FEEDING MEDIA

(71) Applicant: Luyef Biotechnologies INC., Wilmington, DE (US)

(72) Inventors: Stephanie Brain, Santiago (CL); Andrea Villanueva, Santiago (CL); María Soledad Gutierrez, Santiago (CL); Kris Blanchard, Santiago (CL); Pamela Barroso, Santiago (CL)

(73) Assignee: Luyef Biotechnologies INC., Wilmington, DE (US)

(21) Appl. No.: 18/071,742

(22) Filed: Nov. 30, 2022

Related U.S. Application Data
(60) Provisional application No. 63/284,656, filed on Dec. 1, 2021.

(51) Int. Cl. C07K 14/805; C12N 15/80; C12N 9/02; C12N 9/88

(52) U.S. Cl. CPC C07K 14/805; C12N 15/80; C12N 9/0008; C12Y 102/01012; C12N 9/88; C12Y 401/01001

(57) ABSTRACT

A method to express myoglobin in a fungus is described. The method includes optimizing a codon for a fungal host (e.g., Trichoderma reesei), inserting the optimized codon into a plasmid (e.g., a pTrEno plasmid or one of its derivatives), and ultimately collecting the secreted myoglobin from a feeding media in response to the myoglobin being expressed extracellularly. Optionally, the myoglobin is purified.

This application claims priority to U.S. Provisional Patent Application No. 63/284,656, filed on Dec. 1, 2021, which is incorporated by reference for all purposes as if fully set forth herein.

### FIELD OF THE EMBODIMENTS

The field of the invention and its embodiments relate to methods for expressing myoglobin in Trichoderma reesei and collecting the myoglobin from a feeding media.

In accordance with 37 CFR 1.834(c)(1), this application incorporates the sequence listing having the file named 03728LBCS02UTL1US_sequencelisting.xml, which was created and filed via EFS-Web on Apr. 11, 2023 and having a file size of 55 kilobytes.

### BACKGROUND OF THE EMBODIMENTS

Health concerns associated with meat consumption have resulted in a shift to the consumption of plant-based food products. In fact, plant-based food products provide numerous benefits as compared to the animal-based food products they replace. For example, plant-based food products provide health benefits (e.g., less cholesterol or lower levels of saturated fats) and eliminate the negative aspects of animal husbandry, including the environmental impacts of such, the animal confinement, the disruption of maternal-offspring interactions, and the slaughter of animals for their meat. As such, there is a high demand for alternative food products that fulfill the nutritional roles that traditional meat products play.

Production of heme-proteins, such as myoglobin, in bacteria, yeast, microalgae, and plants may be one alternative to meet this demand. Recently, other types of heme-proteins produced in yeast using recombinant technology have been used as an additive in commercial food products to mimic the flavor of meat in their vegetarian food. However, these advancements are difficult to scale. As such, what is needed is a novel method that produces or increases myoglobin expression in a fungus, which is both scalable and sustainable.

Examples of related art are described below:
CN113549561A describes construction and application of a Saccharomyces cerevisiae strain for efficiently synthesizing hemoglobin or myoglobin from different sources.
CN113136349A discloses construction of a Pichia pastoris recombinant strain capable of efficiently expressing myoglobin/hemoglobin from different sources.
US20210070842A1 provides methods and compositions for the expression and secretion of heme-containing polypeptides. Further, this reference describes an alga with a genetic modification, where the genetic modification results in an increase in production of a heme cofactor.
US20200332249A1 describes recombinant microalgae containing one or more polynucleotides encoding iron-complexed proteins, methods of producing the iron-complexed proteins with the microalgae, and edible products formed therefrom.
US20190292555A1 relates to methods of making recombinant target products in transgenic plants in a contained system. This reference describes that the target product can be leghemoglobin (lbc2), a non-symbiotic hemoglobin, an androglobin, a cytoglobin, a globin E, a globin X, a globin Y, a hemoglobin, a myoglobin, an erythrocruorin, a beta hemoglobin, an alpha hemoglobin, a protoglobin, a cyanoglobin, a cytoglobin, a histoglobin, a neuroglobins, a chlorocruorin, a truncated hemoglobin, a truncated 2/2 globin, a hemoglobin 3, a cytochrome, or a peroxidase.
US20180368453A1 describes a meat replica product that includes: (a) a muscle replica comprising 0.1%-5% of a heme-containing protein (e.g., myoglobin), at least one sugar compound and at least one sulfur compound; (b) a fat tissue replica comprising at least one plant oil (e.g., an oil produced by bacteria, algae, archaea or fungi or genetically engineered bacteria, algae, or archaea or fungi); and (c) a connective tissue replica. The muscle replica, fat tissue replica, and connective tissue replica are assembled in a manner that approximates the physical organization of meat, where, upon cooking of the meat replica, two or more volatile compounds that are associated with a cooked meat aroma are produced in an increased amount relative to cooking a meat replica product lacking the heme-containing protein.
US20160340411A1 provides methods and compositions for the expression and secretion of heme-containing polypeptides. The heme-containing polypeptide may be a hemoglobin. The heme-containing polypeptide can be isolated from fungi, such as Saccharomyces cerevisiae, Pichia pastoris, Magnaporthe oryzae, Fusarium graminearum, or Fusarium oxysporum, or from bacteria, such as Escherichia coli, Bacillus subtilis, Synechocistis sp., Aquifex aeolicus, Methylacidiphilum infernorum, or thermophilic bacteria such as Thermophilus.
WO9812913A1 and U.S. Pat. No. 5,959,187A describe a method to produce a transgenic plant engineered to express oxygen-binding proteins such as, for example, hemoglobin, myoglobin, and hemoproteins.
WO2021191913A1 describes a plant cell culture, preferably, carrot cells, which expresses transgenic bovine myoglobin proteins. This unique myoglobin-expressing culture is then transformed into a slurry, which serves as the platform for the production of the plant-based meat substitutes. Furthermore, those meat substitutes are highly nutritious, as they contain beneficial ingredients derived from the plant cells (such as beta-carotene) in addition to high protein (myoglobin) content.
WO2021140488A1 provides a method for preparing a bovine myoglobin that includes constructing a first plasmid containing genes for heme biosynthesis pathway enzymes; constructing a second plasmid containing a gene for Bos taurus myoglobin MYG; constructing a first Escherichia coli production host containing the first plasmid and the second plasmid; and producing the bovine myoglobin by culturing the first Escherichia coli production host. A composition useful as a meat flavor and/or an iron supplement includes the bovine myoglobin prepared in accordance with the method.

One group describes that Trichoderma is an ascomycete fungal genus widely distributed in the soils. In this review, the authors summarized the recombinant proteins produced in Trichoderma since 2014, concerning their origins, hosts, promoters, terminators, signal peptides, yields and commonly used media. Meanwhile, strategies and merging trends in protein production and strain engineering are classified and summarized regarding codon optimization, promoter utilization, transcription factor regulation, post-translational modification and proteolytic degradation inhibition. The authors argue that with state-of-art biotechnologies and more available expression platforms, Trichoderma spp. could be more successful hosts to produce recombinant proteins as desired. See, Huiling Wei, et al., “Recombinant Protein Production in the Filamentous Fungus Trichoderma,” Chinese Journal of Chemical Engineering, February 2021, Volume 30, Pages 74-81, the entire contents of which are hereby incorporated by reference in their entirety.

Another group describes isolation of a cDNA clone for myoglobin mRNA from fetal bovine skeletal muscle using a DNA fragment of human myoglobin exon 2 as a probe. The complete coding sequence of myoglobin as well as the 3′- and part of the 5′-nontranslatable sequences (546 and 66 base pairs, respectively) were determined. The amino acid sequence predicted from the nucleotide sequence was in agreement with that determined in the purified protein from adult bovine cardiac muscle, except for eight amino acid residues: Val-99→IIe, IIe-101→Val, Asn-122→Asp, Ala-124→Gly, Gly-129→Ala, Ala-142→Met, Glu-144→Ala, and Lys-145→Gln. When the myoglobin cDNA was expressed in Saccharomyces cerevisiae under the control of the GAL7 promoter, myoglobin was synthesized as a functionally active holoprotein which bound molecular oxygen reversibly. See, Hideo Shimada, et al., “Expression of Bovine Myoglobin cDNA as a Functionally Active Holoprotein in Saccharomyces cerevisiae,” The Journal of Biochemistry, March 1989, Volume 105, Issue 3, Pages 417-422, the entire contents of which are hereby incorporated by reference in their entirety.

Various similar methods exist in the art. However, their means of operation are substantially different from the present disclosure, as the other inventions fail to solve all the problems taught by the present disclosure.

### SUMMARY OF THE EMBODIMENTS

The present invention and its embodiments relate to methods for expressing myoglobin in Trichoderma reesei and collecting the myoglobin from a feeding media.

A first embodiment of the present invention describes a method to express myoglobin in a fungus. The method includes numerous process steps, such as: optimizing a codon for a fungal host. In examples, the fungal host is a mesophilic and filamentous fungus, and more specifically, is Trichoderma reesei. In some examples, this optimization occurs via software, such as GenScript software or it is manually performed using the preferred codon usage of Trichoderma reesei (Hypocrea jecorina) deposited in public databases. Next, the method also includes inserting the codon-optimized gene into a plasmid. In preferred examples, the plasmid is a pTrPdc or pTrGdp plasmid. Then, the method includes collecting recombinant myoglobin from a feeding media in response to the myoglobin being expressed and secreted extracellularly to the feeding media. Optionally, the method may also include purifying the myoglobin.

Another embodiment of the present invention describes a codon-optimized bovine myoglobin sequence, which includes: a PacI site, an Xbal site, a Kozak sequence, a signal peptide, and a myoglobin opening reading frame (ORF). Specifically, the Kozak sequence is disposed between the PacI site and the signal peptide. Moreover, the myoglobin ORF is disposed between the signal peptide and the Xbal site. Specifically, the myoglobin ORF comprises an ATG codon proximate the signal peptide and a Stop codon proximate the XbaI site. Additionally, the myoglobin ORF is a Bos taurus codon-optimized to a Trichoderma reesei host.

In another embodiment of this invention, a method for producing recombinant bovine myoglobin in T. reesei includes: supplementing the culture media with heme-iron and non heme-iron (inorganic and organic iron supplements) to enhance heme group production and increase production yields of recombinant bovine myoglobin.

In another embodiment of this invention, a method for producing recombinant bovine myoglobin in Trichoderma reesei includes co-expressing enzymes from the porphyrin synthesis pathway; and/or the disruption or overexpression of transcription factors involved in the heme group production.

In another embodiment of this invention, a method for producing recombinant bovine myoglobin in Trichoderma reesei includes the disruption of enzymes of metabolic routes that involve the use of porphyrins or heme B in T. reesei.

In another embodiment of this invention, a method for producing recombinant bovine myoglobin in Trichoderma reesei includes overexpressing, derepressing, repressing or deleting genes and/or transcription factors involved in the oxygen sensing pathway in T. reesei.

In another embodiment of this invention, a method for producing recombinant bovine myoglobin in Trichoderma reesei includes overexpressing, repressing or deleting the coding genes of heme/transporters to increase endogenous heme B in T. reesei cytosol.

### BRIEF DESCRIPTION OF THE DRAWINGS

FIG. 1 depicts a block diagram of a codon-optimized bovine myoglobin sequence, in accordance with embodiments of the present invention.

FIG. 2 depicts a schematic diagram of a plasmid for recombinant expression of a myoglobin derivative in Trichoderma reesei, in accordance with embodiments of the present invention.

FIG. 3 depicts a block diagram of a method to express myoglobin in Trichoderma reesei and collect the myoglobin from a feeding media, in accordance with embodiments of the present invention.

FIG. 4 depicts a block diagram of a method to collect, isolate and purify myoglobin in Trichoderma reesei culture supernatants, in accordance with embodiments of the present invention.

FIG. 5 shows a schematic representation of the parent vector pTrEno-MyoTr used for construction of pTrPdc-MyoTr and pTrGdp-MyoTr constructs.

FIG. 6 shows a diagram of pTrPdc-MyoTr vector.

FIG. 7 shows a diagram of pTrGpd-MyoTr vector.

FIG. 8 shows a diagram of pTrPdcBle-ALAS vector.

FIG. 9 depicts the porphyrin synthesis pathway.

FIG. 10 shows T. reesei transformation plates.

FIG. 11 shows recombinant bovine myoglobin yields in culture supernatants from T. reesei strains, as described in Example 1 (FIG. 11-A) and Example 2 (FIG. 11-B).

FIG. 12 shows recombinant bovine myoglobin yields in culture supernatants from T. reesei cultured with exogenous heme-iron and non heme-iron supplementation, as described in Examples 3 (FIG. 12-A) and 4 (FIG. 12-B).

FIG. 13 shows recombinant bovine myoglobin yields in culture supernatants from T. reesei transformed with pTrPdc-MyoTr (strains 5.1A-51G) and pTrGdp-MyoTr (strains 5.2A-5.2F), as described in Example 5.

FIG. 14 shows results from ELISA detection of recombinant bovine myoglobin in culture supernatants from T. reesei cotransformed with pTrGdp-MyoTr and pTrBle-ALAS (14-A), as described in Example 7. FIG. 14-B shows the effects of iron supplementation (heme and non heme) in myoglobin yields in a selected strain (7E), as described in Example 8.

### DESCRIPTION OF THE PREFERRED EMBODIMENTS

The preferred embodiments of the present invention will now be described with reference to the drawings. Identical elements in the various figures are identified with the same reference numerals.

Reference will now be made in detail to each embodiment of the present invention. Such embodiments are provided by way of explanation of the present invention, which is not intended to be limited thereto. In fact, those of ordinary skill in the art may appreciate upon reading the present specification and viewing the present drawings that various modifications and variations can be made thereto.

### Myoglobin and Hemoglobin

Iron is an essential micronutrient that plays an essential role for oxygen transport in the body, and is an important constituent of hemoglobin, myoglobin, cytochrome, iron/sulfur protein and other biomolecular structures, such as ferritin and transferrin. Iron is not synthesized in the body and thus must be acquired entirely through intake.

Iron deficiency and resulting anemia being described as a worldwide health concern. To remedy this problem, some groups have developed methods of iron biofortification of crop species, using plant breeding and genetic engineering. See, H. E. Bouis, et al., “Improving Nutrition Through Biofortification: A Review of Evidence From HarvestPlus, 2003 through 2016,” Glob. Food., 2017, Sec. 12, Pages 49-58; and M. Garg, et al., “Biofortified Crops Generated by Breeding, Agronomy, and Transgenic Approaches are Improving Lives of Millions of People Around the World,” Front. Nutr., 2018, Vol. 5, doi: 10.3389/fnut.2018.00012, the entire contents of which are hereby incorporated by reference in their entirety.

Myoglobin is a heme-protein in the muscle of vertebrates that serves important functions in the oxygenation of tissues and serves as a regulator in nitric oxide signaling. See, Magnus L. R. Carlsson, et al., “Plant based Production of Myoglobin—A Novel Source of the Muscle Heme-Protein,” Scientific Reports, 2020, Vol. 10, Article No. 920; B. A. Wittenberg, et al., “Myoglobin-Mediated Oxygen Delivery to Mitochondria of Isolated Cardiac Myocytes,” Proc. Natl. Acad. Sci. USA, 1987, Vol. 84, Pages 7503-07; and J. B. Wittenberg, et al., “Myoglobin Function Reassessed,” J. Exp. Biol., 2003, Vol. 206, Pages 2011-20, the entire contents of which are hereby incorporated by reference in their entirety. Hemoglobin is the iron-containing oxygen-transport metalloprotein in the red blood cells of some vertebrates, as well as the tissues of some invertebrates. In fact, both hemoglobin and myoglobin are an important nutritional source of bioavailable iron.

Hemoglobin and myoglobin are heme-proteins. Heme-proteins are defined a proteins that contain a heme prosthetic group, which allows oxygen carrying, oxygen reduction, electron transfer, and other processes. The heme group consists of an iron cation bound at the center of the conjugate base of the porphyrin ring, a planar dianionic, tetradentate ligand. The porphyrin ring is a heterocyclic macrocycle organic compounds, composed of four modified pyrrole subunits interconnected at a carbon atoms via methine bridges (═CH—). Thus, the porphyrin ring has 4 nitrogen atoms that bind to the iron, leaving two other coordination positions of the iron available for bonding to the histidine of the protein and a divalent atom. Heme group is bound to the protein either covalently or non-covalently or both. Generally, heme is attached to the apoprotein through a single coordination bond between the heme iron and an amino-acid side-chain. The iron bound to heme is typically Fe²⁺ or Fe³⁺.

Compared to hemoglobin, myoglobin has a higher affinity for oxygen and does not have cooperative binding with oxygen like hemoglobin does. On the other hand, both hemoglobin and myoglobin have a conserved histidine residue that binds to the iron center of the heme moiety. In myoglobin, a proximal histidine group (His-93) is directly attached to iron in the heme moiety, and a distal histidine group (His-64) hovers near the opposite face. The distal imidazole moieties in the histidine groups are available to interact with the substrate O₂. This interaction encourages the binding of O₂, but not carbon monoxide (CO), which prefers oxygen to carbon monoxide despite the fact that CO binds about 240× more strongly than O₂.

### Myoglobin Expression

Other groups have taken different approaches. Numerous groups have developed methods aimed at increasing myoglobin expression in yeast, such as Pichia pastoris (Komagataella phaffii). Pichia pastoris is methylotrophic yeast that can grow using methanol as the sole carbon source. P. pastoris has been established as a protein production host. See, Ryosuke Yamada, “Chapter 17 —Pichia pastoris-Based Microbial Cell Factories,” Microbial Cell Factories Engineering for Production of Biomolecules, 2021, Pages 335-344; and B. Zhang, et al., “Efficient Secretory Expression and Purification of Food-Grade Porcine Myoglobin in Komagataella phaffii,” Journal of Agricultural and Food Chemistry, 2021, Vol. 69, Issue 35, Pages 10235-10245, the entire contents of which are hereby incorporated by reference in their entirety. In fact, one group, Motif FoodWorks, Inc., submitted a Food and Drug Administration (FDA) Generally Recognized as Safe (GRAS) notification for myoglobin that is being expressed in a genetically engineered yeast strain to mimic flavors associated with cooked ground meat. Another group, Impossible Foods, Inc., submitted FDA GRAS Notification No. 737 For Soy Leghemoglobin Protein Preparation Derived From P. pastoris. See, also, WO 2016/183163 A1, published on Nov. 16, 2016, the entire contents of which are hereby incorporated by reference in their entirety.

Moreover, other groups have developed methods related to myoglobin expression in plants. See, M. L. Carlsson, et al., “Plant-Based Production of a Myoglobin—A Novel Source of the Muscle Hemeprotein,” Scientific Reports, 2020, Vol. 10, Issue 1, Pages 1-10, the entire contents of which are hereby incorporated by reference in their entirety. Others have developed methods of myoglobin expression in microalgae. See, U.S. Published Patent Application S/N 2020/0332249 A1, published on Oct. 22, 2020, the entire contents of which are hereby incorporated by reference in their entirety. Some groups have even developed methods for increasing production of heme proteins in fungal variants. See, JP2001526037A, published on Dec. 18, 2001 and JP2000512151A, published on Sep. 19, 2000, the entire contents of which are hereby incorporated by reference in their entirety.

### Fungal Host

Trichoderma is an ascomycete fungal genus widely distributed in soil. This genus comprises many species of filamentous fungi that form mutualistic endophytic relationships with several plant species. Examples include the biocontrol species such as Trichoderma harzianum, T. viride, T. koningii, and T. atroviride and the soft-rot ascomycete Trichoderma reesei.

Trichoderma reesei (T. reesei), is a mesophilic and filamentous fungus and is an anamorph of the fungus Hypocrea jecorina (previously classified as T. longibrachiatum). T. reesei can secrete large amounts of cellulolytic enzymes. Moreover, microbial cellulases have industrial application in the conversion of cellulose into glucose. T. reesei is an important commercial and industrial microorganism due to its cellulase production ability. T. reesei is able to secrete up to 100 g/L of homologous proteins into the culture supernatants, mainly consisting of cellulase enzymes. The highest reported yields for heterologous proteins expressed in T. reesei are those of foreign fungal and bacterial proteins that have reached g/L levels, and the highest yields for mammalian proteins are in the order of μg/L. The high extracellular secretion capability and eukaryotic post-translational modification machinery make T. reesei an interesting host for several applications. See, Huiling Wei, et al., “Recombinant Protein Production in the Filamentous Fungus Trichoderma,” Chinese Journal of Chemical Engineering, February 2021, Vol. 30, Pages 74-81, the entire contents of which are hereby incorporated by reference in their entirety.

T. reesei is generally considered a safe production host microorganism. Many T. reesei enzymes have obtained the generally recognized as safe (GRAS) status by the U.S. Food and Drug Administration. See, Nevalainen et al., “On The Safety of Trichoderma reesei,” Journal of Biotechnology, June 1994, Vol. 37, Pages 193-200, the entire contents of which are hereby incorporated by reference in their entirety.

T. reesei strains used for the expression of recombinant bovine myoglobin are QM6a (ATCC 13631), QM9414 (ATCC 26921). QM9136 (ATCC 26920), Rut-C30 (ATCC 56765, NRRL 11460), Rut-NG14 (ATCC 56767, NRRL 11485), Rut-D4 (ATCC 56766, NRRL 11480), X-31 (ATCC 66587, NRRL 15502), TU-6 (ATCC MYA-256, a uridine auxotroph), MCG 77 (ATCC 56764, NRRL 11236), MCG 80 (ATCC 60787, NRRL 12368), NRRL 12368, QM 9123 (ATCC 24449, NRRL 3653), M5 (ATCC 58350) 4065 MG5 (ATCC 46481), M6 (ATCC 58351), MHC22 (ATCC 58353), PC-1-4 (ATCC 66588, NRRL 15499), VKH1 234 (ATCC 46480), PC-3-7 (ATCC 66589, NRRL 15500), among others. Also, protease deficient cell strains derived from the listed above can be used in the present invention. However, the invention is not intended to be limited to these specific cells, as in other embodiments, other cells may be used.

### Development of Expression Constructs and Regulatory Sequences Involved

In the process of producing recombinant bovine myglobin in T. reesei, basic genetic strategies to enhance protein yields are used, including: (1) the optimization of codon usage, (2) the introduction of multiple copies of the gene of interest, (3) the use of strong constitutive or inducing promoters, (3) the co-expression of transcription factors, (4) the use of efficient secretion signals, (5) the use of gene fusions, and (6) the use of protease deficient strains.

The recombinant bovine myoglobin of the present invention is also described as “myoglobin derivative”, and includes variants thereof having at least 70%, 75%, 80%, 85%, 90%, 95%, 99%, or 99.5% identity to the amino acid sequence of SEQ ID NO: 1, but not limited thereto. The amino acid sequence identity is defined herein as the percentage of amino acid residues in a candidate sequence that are identical with the amino acid residues in the globin sequence, after aligning the sequence in the same reading frame and introducing gaps, if necessary, to achieve the maximum percent sequence identity, and not considering any conservative substitutions as part of the sequence identity.

In many cases, multiple rounds of engineering are needed to achieve a satisfactory expression level. Another strategy is to manipulate the nucleotide sequences of the gene of interest or the promoter/terminator controlling its expression.

The term “regulatory sequence” as used herein is meant to include all components that direct the expression of a coding sequence for recombinant bovine myoglobin in a fungal cell under conditions compatible with the regulatory sequence. Expression will be understood to include any step associated with production of the polypeptide, including but not limited to transcription, post-transcriptional modification, translation, post-translational modification, and secretion. The regulatory sequence may be native to the fungal cell, may be obtained from another source, or may be a combination of native and foreign regulatory sequences. The foreign regulatory sequence may be replaced by, or in addition to, the native regulatory sequence to obtain enhanced production of the recombinant bovine myoglobin. Such regulatory sequences include, but are not limited to, a leader, a propeptide sequence, a polyadenylation sequence, a promoter, a signal peptide sequence, and a transcription terminator. For expression under the direction of a regulatory sequence, the nucleic acid sequence to be used in accordance with the present invention has its regulation regulated such that expression is achieved under conditions compatible with the regulatory sequence.

The term “coding sequence” as defined herein is a sequence that is transcribed into mRNA and translated into recombinant bovine myoglobin when placed under the restrictions of the regulatory sequences described above. The boundaries of the coding sequence generally consist of the ATG start codon located just upstream of the open reading frame at the 5′ end of the mRNA and the 3′ end determined by the transcription terminator sequence located immediately downstream of the open reading frame at the 3′ end. Coding sequences include, but are not limited to, DNA, cDNA, and recombination spreading sequences.

The term “operably linked” is defined herein as a configuration in which a regulatory sequence is properly located at a position relative to a coding sequence of its DNA sequence such that the regulatory sequence directs production of the polypeptide.

Other regulatory sequence may be a suitable promoter sequence that is a nucleic acid sequence recognized by the fungal cell for expression of the codon-optimized bovine myoglobin gene sequence. The promoter sequence contains transcriptional and translational regulatory sequences that mediate the expression of the recombinant bovine myoglobin. The promoter can be any promoter sequence showing transcriptional activity in the selected fungal cell and can be obtained from a gene native or exogenous to the fungal cell. Suitable promoters for directing transcription of recombinant bovine myoglobin sequence in the fungal cell include hybrids of promoters from different genes, promoters and variants, truncated and hybrid promoters thereof. The transcription of the recombinant bovine myoglobin sequence can be performed using one single promoter or multiple promoters inducible under the same conditions but only partly sharing the regulatory factors. Suitable promoters (constitutive or inducible) for directing transcription of recombinant bovine myoglobin sequence in a filamentous fungal cell are: Aspergillus nidulans glyceraldehyde-3-phosphate dehydrogenase (P_{gpdAd}), A. nidulans (1,4)-β-D-arabinoxylan-arabinofuranohydrolase (P_{axhA}), A. nidulans alcohol deshydrogenase (P_{alcC}), A. nidulans acetamidase (P_{ace2}); pre region from Aspergillus niger neutral α-amylase gene, A. niger protein kinase (P_{pkiA}), A. niger alcohol dehydrogenase (P_{adhA}), A. niger glutamate dehydrogenase (P_{gdhA}), A. niger glyceraldehyde-3-phosphate dehydrogenase (P_{gpdA}), A. niger glucoamylase A (P_{glaA}), A. niger benzoate parahydroxylase A (P_{bphA}), A. niger synthetic core promoters; Aspergillus oryzae TAKA amylase (P_{amyB}), A. oryzae alkaline protease (Pₐₗₚ), A. oryzae triose phosphate isomerase (Pₜₚᵢ), A. oryzae oxidoreductase (P_{kojA}), A. oryzae thiamine thiazole synthase (P_{thiA}); Cryptococcus neoformans actin promoter (P_{act}); Neurospora crassa P_{qa2}, and promoters from N. crassa photoreceptors White Collar-1 (WC-1) and White Collar-2 (WC-2); Penicillium chrysogenum phosphoglycerate kinase gene (P_{pgkA}), P. chrysogenum phosphate-repressible acid phosphatase (P_{phoA}), P. chrysogenum endo-xylanase (P_{xylP}), P. chrysogenum synthetase (P_{pcbC}); Pichia pastoris (P_{gap1}); Rhizomucor miehei aspartate proteinase (P_{rmproA}); Saccharomyces cerevisiae phosphoglycerate kinase (P_{pgk1}), S. cerevisiae glyceraldehyde 3-phosphate dehydrogenase (P_{gdp}), S. cerevisiae cytochrome c isoform 1 (P_{cyc1}), S. cerevisiae acid phosphatase 1 (Pₚₕₒ₅), S. cerevisiae alcohol dehydrogenase 1 (P_{adh1}), S. cerevisiae triose phosphateisomerase (Pₜₚᵢ₁), S. cerevisiae hexose transporter (Pₕₓₜ₇), S. cerevisiae pyruvate kinase 1 (P_{pyk1}), S. cerevisiae triose phosphate dehydrogenase (P_{tdh3}), S. cerevisiae P_{gal1}, P_{gal2}, P_{gal3}, P_{gal4}, P_{gal5}, P_{gal6}, P_{gal7}, P_{gal10}, and P_{gal80}; Yarrowia lipolytica transcription elongator factor 1-alpha (P_{tef1}), Y. lipolytica acyl-CoA oxidase 2 transporter (Pₒₓ₂); Trametes versicolor glyceraldehyde-3-phosphate dehydrogenase (P_{gpd}); T. reesei cellobiahydrolase I or Cel7A (P_{cbh1}) described in GenBank No. D86235, T. reesei cellobiohydrolase II or Cel6A (P_{cbh2}), T. reesei enolase (Pₑₙₒ₁), T. reesei transcription elongator factor 1-alpha (P_{tef1}), T. reesei hydrophobin 1 (P_{hfb1}), T. reesei ribosomal protein (Pᵣₚ₂), T. reesei HEX1 (Pₕₑₓ₁), T. reesei pyruvate decarboxylase (P_{pdc}), T. reesei glyceraldehyde-3-phosphate dehydrogenase (P_{gpd}), T. reesei triose phosphate isomerase (Pₜₚᵢ), T. reesei pyruvate deshydrogenase (P_{pda}), T. reesei hydrophobin II core promoter (P_{hfb2cp}), T. reesei ketoglutarate dehydrogenase (P_{kdh}), T. reesei alcohol dehydrogenase (P_{adh}), T. reesei aldolase (P_{fba}), T. reesei pyruvate kinase (P_{pyk}), T. reesei alpha-ketoglutarate dehydrogenase (P_{kdh}), T. reesei citrate synthase (P_{cit}), T. reesei aldehyde dehydrogenase II (P_{ald2}), T. reesei xylanase II (P_{xyn2}), T. reesei endoglucanase I or Cel5A (P_{egl1}), T. reesei endoglucanase II (P_{egl2}), T. reesei ribosomal protein II (Pᵣₚ₂), and T. reesei glucokinase (P_{glk}), T. reesei L-methionine repressible promoters (Pₘₑₜ₃), T. reesei copper transporter promoter (P_{tcu1}), among others. Other suitable promoters also include a selection of established artificial expression systems for T. reesei, such as pristinamycin on/off system, erythromycin on/off system, Tet-on/off system, the temperature inducible gene regulation (TIGR) system, cumate gene-switch (4-isopropylbenzoic acid), or biotin triggered genetic switch. Also, it can be performed using a light-sensitive gene expression regulation with light by fusing S. cerevisiae Gal4 DNA-binding domain to N. crassa blue-light photoreceptor to the Vivid and the H. simplex VP16 activation domain, or using promoters from T. reesei light photoreceptors BLR1, BLR2 (homologs of N. crassa WC-1 and WC-2), and ENV1 (ortologue of N. crassa VIVID). However, the invention is not intended to be limited to these specific promoters, as in other embodiments, other promoters may be used.

Suitable transcription factors useful for the transcription of recombinant bovine myoglobin gene in T. reesei include: CRE1 (accounts for the repression of genes responsible for the entry of cellulase inducers into the cell), XYR1 (master activator for cellulases and xylanases), ACE1 (a Cys2His2-type zing finger protein, ACE2 (a zinc binuclear cluster protein), ACE3 (an activator that enhances cellulase production and decrease xylanase activity), ACE4, RCE1 (a zinc binuclear cluster protein that acts as a transcriptional repressor), ARA1 (involved in the utilization of D-galactose and L-arabinose). Also, analogs of S. cerevisiae transcriptions factors (MIG1, MED2, PGD1, NRG1, MIG2, CTI6, NGG1, SPT3, SPT8, GCN5, ADA2, NPH6B, CSR2, SNF1, SNF4, XYLR, and MTH1), can be involved.

Another regulatory sequence may be a suitable transcription terminator sequence, a sequence recognized by the fungal cell to terminate transcription. The terminator sequence is operably linked to the 3′ end of the second nucleic acid sequence encoding the bovine myoglobin. The terminator sequence may be native or foreign to the second nucleic acid sequence encoding the bovine myoglobin. Any terminator that is functional in the selected mutant fungal cell appears to be useful in the present invention. Preferred terminators for filamentous fungal host cells are A. oryzae TAKA amylase terminator, A. niger glucoamylase (T_{alaA}), A. nidulans anthranilate synthase (Tₐₛₐ₁), A. niger α-glucosidase (T_{agdA}), A. nidulans T_{trpC} terminator, A. nidulans AN7354.2 terminator (40S ribosomal subunit protein), C. neoformans Trp1 terminator (Tₜᵣₚ₁), S. cerevisiae ADH1 gene terminator (T_{adh1}), S. cerevisiae iso-1-cytochrome c gene terminator (T_{cyc1}), T. reesei T_{tef1} terminator, T. reesei T_{cbh1} terminator, T. reesei T_{cbh2} terminator, T. reesei T_{trpC} terminator, T. reesei T_{pgi1} terminator

To enhance the production of recombinant heterologous proteins using microbial cells, it is widely described the addition of a translational enhancer sequence. Sometimes it is a eukaryotic sequence, such as a Kozak consensus sequence or other sequence (e.g., hydroid polyp sequence, GenBank accession No. U07128). A translational enhancer sequence sometimes is from a 5′ UTR of a plant virus, such as Tobacco Mosaic Virus (TMV), Alfalfa Mosaic Virus (AMV); Tobacco Etch Virus (ETV) and Pea Seed Borne Mosaic Virus, for example.

In certain embodiments, the addition of a Kozak sequence upstream of the gene sequence of the target protein is included. The purine base located 3 bases upstream of the start codon (AUG) is translated with higher efficiency when either A or G is used. In heterologous protein production it is necessary to individually examine which Kozak sequence is suitable for the host cell. If this Kozak sequence is not optimized, the translation efficiency of the protein may be reduced and the productivity may be reduced.

In the present invention, the Kozak DNA sequence is included in SEQ ID NO: 1, and is preferably adjacent to the codon-optimized gene of interest. The gene of interest can be the codon-optimized bovine myoglobin gene for T. reesei (SEQ ID NO: 1), or any enzyme from the porphyrin synthesis pathway, such as the DNA sequences Δ-aminolevulinate synthase (5-aminolevulinate synthase) or ALAS (SEQ ID NO: 3), Δ-aminolevulinic acid dehydratase or ALAD (SEQ ID NO: 5), porphobilinogen deaminase or PGDB (SEQ ID NO: 7), uroporphyrinogen synthase (uroporphyrinogen III synthase) or UROS (SEQ ID NO: 9), uroporphoryinogen decarboxylase or UROD (SEQ ID NO: 11) coproporphyrinogen oxidase or CPO (SEQ. ID NO: 13), protoporphyrinogen oxidase or PPO (SEQ ID NO: 15), or ferrochelatase or FC (SEQ ID NO: 17). Other sequences of interest include uroporphyrinogen III decarboxylase or UROD, coproporphyrinogen III oxidase or CPO, protoporphyrinogen IX oxidase or PPO, and/or other ferrochelatase(s) or FC (SEQ ID NO 17). The Kozak consensus DNA sequence can be CCAACATGAGA, CCACCATGGC, CACAATGGC, ACAATGG, or GCCAACATGG. In a preferred embodiment, the Kozak sequence CCAACATGAGA allowed high efficiency in translation and the productivity of recombinant bovine myoglobin was improved.

The full list of sequences are as shown in the following Table 1A.

TABLE 1A

| SEQ ID NO: | Sequence |
|---|---|
| 1 | DNA sequence of codon-optimized bovine myoglobin |
| | gene for T. reesei |
| 2 | Amino Acid sequence of bovine myoglobin |
| 3 | DNA sequence of D-aminolevulinate synthase |
| 4 | Amino Acid sequence of D-aminolevulinate synthase |
| 5 | DNA sequence of D-aminolevulinic acid dehydratase |
| 6 | Amino Acid sequence of D-aminolevulinic acid dehydratase |
| 7 | DNA sequence of porphobilinogen deaminase |
| 8 | Amino Acid sequence of porphobilinogen deaminase |
| 9 | DNA sequence of uroporphoryinogen synthase |
| 10 | Amino Acid sequence of uroporphoryinogen synthase |
| 11 | DNA sequence of uroporphoryinogen decarboxylase |
| 12 | Amino Acid sequence of uroporphoryinogen decarboxylase |
| 13 | DNA sequence of coproporphyrinogen oxidase |
| 14 | Amino Acid sequence of coproporphyrinogen oxidase |
| 15 | DNA sequence of protoporphyrinogen oxidase |
| 16 | Amino Acid sequence of protoporphyrinogen oxidase |
| 17 | DNA sequence of ferrochelatase |
| 18 | Amino Acid Sequence of ferrochelatase |
| 19 | DNA sequence of SEQF primer |
| 20 | DNA sequence of SEQR primer |
| 21 | DNA sequence of Ppdc_SbfI-F primer |
| 22 | DNA sequence of Ppdc_PacI-R primer |
| 23 | DNA sequence of Pgpd_SbfI-F primer |
| 24 | DNA sequence of Pgpd_PacI-R primer |
| 25 | DNA sequence of ble_Ppgk-F primer |
| 26 | DNA sequence of ble-Tcbh1-R primer |
| 27 | DNA sequence of Tcbh1-ble-F primer |
| 28 | DNA sequence of Ppgk-ble-R primer |
| 29 | DNA sequence of ALAS-PacI-F primer |
| 30 | DNA sequence of ALAS-XbaI-R primer |
| 31 | DNA sequence of ALAS-F primer |
| 32 | DNA sequence of ALAS-R primer |

By nucleic acid encoding ALAS (or variants thereof) is meant a nucleic acid (or polynucleotide) having at least 40% nucleic acid sequence identity to SEQ ID NO: 3 in which the encoded ALAS has 5-aminolevulinate synthase activity. For example, a nucleic acid encoding ALAS can have at least 45%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98% or even at least 99% amino acid sequence identity to SEQ ID NO: 4. In certain embodiments, the nucleic acid encoding ALAS is 100% identical to SEQ ID NO: 3. In additional embodiments, a nucleic acid encoding ALAS hybridizes under stringent conditions to a nucleic acid having a sequence complementary to SEQ ID NO: 3.

By ALAS is meant an enzyme having at least 80% amino acid sequence identity to SEQ ID NO: 4 and having 5-aminolevulinate synthase activity (as described above). For example, a ALAS having 5-aminolevulinate synthase activity can have at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even 100% amino acid sequence identity to SEQ ID NO: 4.

By nucleic acid encoding ALAD (or variants thereof) is meant a nucleic acid (or polynucleotide) having at least 40% nucleic acid sequence identity to SEQ ID NO: 5 in which the encoded ALAD has 5-aminolevulinate dehydratase activity. For example, a nucleic acid encoding ALAD can have at least 45%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 6. In certain embodiments, the nucleic acid encoding ALAD is 100% identical to SEQ ID NO: 5. In additional embodiments, a nucleic acid encoding ALAD hybridizes under stringent conditions to a nucleic acid having a sequence complementary to SEQ ID NO: 5.

By ALAD is meant an enzyme having at least 80% amino acid sequence identity to SEQ ID NO: 6 and having 5-aminolevulinate dehydratase activity (as described above). For example, a ALAD having 5-aminolevulinate dehydratase activity can have at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even at least 100% amino acid sequence identity to SEQ ID NO: 6.

By nucleic acid encoding PGBD (or variants thereof) is meant a nucleic acid (or polynucleotide) having at least 40% nucleic acid sequence identity to SEQ ID NO: 7 in which the encoded PGBD has porphobilinogen deaminase activity. For example, a nucleic acid encoding PGBD can have at least 45%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 8. In certain embodiments, the nucleic acid encoding PBGD is 100% identical to SEQ ID NO: 7. In additional embodiments, a nucleic acid encoding PBGD hybridizes under stringent conditions to a nucleic acid having a sequence complementary to SEQ ID NO: 7.

By PBGD is meant an enzyme having at least 80% amino acid sequence identity to SEQ ID NO: 8 and having porphobilinogen deaminase activity (as described above). For example, a PBGD having porphobilinogen deaminase activity can have at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even at least 100% amino acid sequence identity to SEQ ID NO: 8.

By nucleic acid encoding UROS (or variants thereof) is meant a nucleic acid (or polynucleotide) having at least 40% nucleic acid sequence identity to SEQ ID NO: 9 in which the encoded UROS has uroporphyrinogen III synthase activity. For example, a nucleic acid encoding UROS can have at least 45%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 10. In certain embodiments, the nucleic acid encoding UROS is 100% identical to SEQ ID NO: 9. In additional embodiments, a nucleic acid encoding UROS hybridizes under stringent conditions to a nucleic acid having a sequence complementary to SEQ ID NO: 9.

By UROS is meant an enzyme having at least 80% amino acid sequence identity to SEQ ID No. 10 and having uroporphyrinogen III synthase activity (as described above). For example, a UROS having uroporphyrinogen III synthase activity can have at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even at least 100% amino acid sequence identity to SEQ ID NO: 10.

By nucleic acid encoding UROD (or variants thereof) is meant a nucleic acid (or polynucleotide) having at least 40% nucleic acid sequence identity to SEQ ID NO: 11 in which the encoded UROD has uroporphyrinogen III decarboxylase activity. For example, a nucleic acid encoding UROD can have at least 45%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 12. In certain embodiments, the nucleic acid encoding UROD is 100% identical to SEQ ID NO: 11. In additional embodiments, a nucleic acid encoding UROD hybridizes under stringent conditions to a nucleic acid having a sequence complementary to SEQ ID NO: 11.

By UROD is meant an enzyme having at least 80% amino acid sequence identity to SEQ ID No. 12 and having uroporphyrinogen III decarboxylase activity (as described above). For example, a UROD having uroporphyrinogen III decarboxylase activity can have at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even at least 100% amino acid sequence identity to SEQ ID NO: 12.

By nucleic acid encoding CPO (or variants thereof) is meant a nucleic acid (or polynucleotide) having at least 40% nucleic acid sequence identity to SEQ ID NO: 13 in which the encoded CPO has coproporphyrinogen III oxidase activity. For example, a nucleic acid encoding CPO can have at least 45%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 14. In certain embodiments, the nucleic acid encoding CPO is 100% identical to SEQ ID NO: 13. In additional embodiments, a nucleic acid encoding CPO hybridizes under stringent conditions to a nucleic acid having a sequence complementary to SEQ ID NO: 13.

By CPO is meant an enzyme having at least 80% amino acid sequence identity to SEQ ID No. 14 and having coproporphyrinogen III oxidase activity (as described above). For example, a CPO having coproporphyrinogen III oxidase activity can have at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even at least 100% amino acid sequence identity to SEQ ID NO: 14.

By nucleic acid encoding PPO (or variants thereof) is meant a nucleic acid (or polynucleotide) having at least 40% nucleic acid sequence identity to SEQ ID NO: 15 in which the encoded PPO has protoporphyrinogen IX oxidase activity. For example, a nucleic acid encoding PPO can have at least 45%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 16. In certain embodiments, the nucleic acid encoding PPO is 100% identical to SEQ ID NO: 15. In additional embodiments, a nucleic acid encoding PPO hybridizes under stringent conditions to a nucleic acid having a sequence complementary to SEQ ID NO: 15.

By PPO is meant an enzyme having at least 80% amino acid sequence identity to SEQ ID No. 16 and having protoporphyrinogen IX oxidase activity (as described above). For example, a PPO having protoporphyrinogen IX oxidase activity can have at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even at least 100% amino acid sequence identity to SEQ ID NO: 16.

By nucleic acid encoding FC (or variants thereof) is meant a nucleic acid (or polynucleotide) having at least 40% nucleic acid sequence identity to SEQ ID NO: 17 in which the encoded FC has ferrochelatase activity. For example, a nucleic acid encoding FC can have at least 45%, at least 50%, at least 60%, at least 65%, at least 70%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or even at least 99% amino acid sequence identity to SEQ ID NO: 18. In certain embodiments, the nucleic acid encoding FC is 100% identical to SEQ ID NO: 17. In additional embodiments, a nucleic acid encoding FC hybridizes under stringent conditions to a nucleic acid having a sequence complementary to SEQ ID NO: 17.

By FC is meant an enzyme having at least 80% amino acid sequence identity to SEQ ID No. 18 and having ferrochelatase activity (as described above). For example, a FC having ferrochelatase activity can have at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or even at least 100% amino acid sequence identity to SEQ ID NO: 18.

A target nucleic acid sometimes can comprise a chimeric nucleic acid (or chimeric nucleotide sequence), which can encode a chimeric protein (or chimeric amino acid sequence). The term “chimeric” as used herein refers to a nucleic acid or nucleotide sequence, or encoded product thereof, containing sequences from two or more different sources. Suitable sources include, but not limited to, a sequence from a nucleic acid, nucleotide sequence, gene, a ribosomal nucleic acid, RNA, DNA, a chromosome, a regulatory nucleotide sequence (e.g., promoter, URL, enhancer, repressor and the like), a coding nucleic acid, nucleic acid linker, nucleic acid tag, amino acid sequence, peptide, polypeptide, protein, and organism. A chimeric molecule can include a sequence of contiguous nucleotides or amino acids from a source including, but not limited to, homolog, ortholog, and paralog. A chimeric molecule can include a sequence from a virus, prokaryote, eukaryote, nucleic acid linkers, nucleic acid tags, the like and combinations thereof. In certain embodiments, the nucleotide sequences or DNA segments can be native or wild type sequences, mutant sequences or engineered sequences. In certain embodiments, a chimeric nucleic acid or nucleotide sequence encodes the same activity as the activity encoded by the source nucleic acids or nucleotide sequences. In some embodiments, a chimeric nucleic acid or nucleotide sequence has a similar or the same activity but it is altered (e.g., increased, decreased). In certain embodiments, a chimeric nucleic acid or nucleotide sequence encodes a different activity, and in some embodiments a chimeric nucleic acid or nucleotide sequences encodes a chimeric activity (e.g., a combination of two or more activities).

Secretion of intracellular proteins is a complex aspect of protein production in microbial expression systems. The information required to identify a compactly folded protein structure is present in the amino acid sequence of the protein. Nonetheless, in vivo protein folding occurs in an environment where many proteins are concentrated, and intermolecular aggregation reactions compete with the intramolecular folding process. Secretory proteins devoid of further address tags in their sequence are by default secreted to the external environment. The first step in the eukaryotic secretion pathway is the transport of nascent polypeptides across the endoplasmic reticulum (ER) in an elongated state. Polypeptide folding and assembly occur in the ER through the secretory pathway. However, during overexpression of heterologous proteins usually result in proteins not properly secreted. Although a “signal sequence”, “signal peptide” or “localization signal sequence” is not responsible for the final destination of the mature protein, it aids the proper folding through the secretory pathway.

A “signal sequence”, “signal peptide” or “localization signal sequence” comprises a sequence that localizes a translated protein or peptide to a component in a system. The signal sequence contains a sequence of amino acids that directs the protein to the secretory system of the host cell, resulting in secretion of the protein from the host cell into the medium in which the host cell is growing. The signal sequence is cleaved from the fusion protein prior to secretion of the protein. A signal sequence often is incorporated at the N-terminus of the target protein or target peptide, and sometimes is incorporated at the C-terminus. The signal sequence employed may be endogenous or non-endogenous to the host cell and, in certain embodiments, may be signal sequence of a protein that is known to be highly secreted from a host cell. A signal sequence in some embodiments localizes the translated protein or peptide to a cell membrane or the extracellular space. In some embodiments, the regulatory sequences in the present invention may be from different genus or species of fungi, including but not limited to, Acremonium spp, Aspergillus aculeatus, A. niger var. awamori, A. flavus, A. fumulus, A. nidulans, A. niger, A. oryzae, A. versicolor, Cephalosporum spp., Dactylum spp., Fusarium spp., Hansenula spp., Humicola spp., Kluyveromyces spp., Neurospora spp. Neocallimastix spp., Penicillium spp., Phanerochaete spp., Pichia spp., Rhizopus spp., Saccharomyces spp., Schizophyllum spp., Schizosaccharomyces spp., Trametes spp., Trichoderma spp., and Yarrowia spp.

Examples of signal sequences include, but are not limited to, mitochondrial targeting signal (e.g., amino acid sequence that forms an amphipathic helix); peroxisome targeting signal (e.g., C-terminal sequence in YFG from S. cerevisiae), a secretion signal (e.g., N-terminal sequences from invertase, alpha-mating factor, PHO5 and SUC2) in S. cerevisiae, pectate lyase signal sequence (e.g., U.S. Pat. No. 5,846,818), A. oryzae TAKA amylase gene signal peptide, A. niger neutral amylase gene signal peptide, A. niger var. awamori glucoamylase (GlaA) signal peptide, T. reesei glucoamylase (GlaA), cellobiohydrolase I (CBH1), cellobiohydrolase II (CBH2), and NSP24 aspartic protease (NSP24) signal peptides, B. brevis signal sequence (e.g., U.S. Pat. No. 5,232,841), and P. pastoris signal sequence (e.g., U.S. Pat. No. 5,268,273). Some examples of signal sequences used in the present invention include, but are not limited to T. reesei CBH1, CBH2, A. niger GlaA, and A. niger var. awamori NSP24 signal peptides.

The T. reesei CBH1 signal sequence, linker, and catalytic site come from cellobiohydrolase 1 (CBH1) coding gene. This signal sequence allows folding to the catalyst and binding site, so the foreign polypeptide is secreted in T. reesei as a fusion with a catalytic site plus a linker region of CBH1. The linker may be used to connect the catalytic site of the T. reesei enzyme and the desired polypeptide. However, any suitable linker may be used in the present invention as long as the linker is extended between the independently folded regions to form a semi-rigid spacer. Such linker regions are found in several proteins, particularly hydrolases. In another embodiment, the invention comprises T. reesei CBH2 signal sequence to secrete recombinant bovine myoglobin. In another embodiment, the invention comprises A. niger var. awamori alpha-amylase signal sequence to secrete recombinant bovine myoglobin. In another embodiment, the invention comprises A. niger glucoamylase signal sequence to secrete recombinant bovine myoglobin.

Gene fusions can be used to enhance secretion of proteins into the feeding medium and improve protein yields of non-fungal proteins. These gene fusions improve import and trafficking through the secretory pathway, making the heterologous protein less susceptible to stick to the cell wall and act as a shield against proteolysis. The heterologous gene is linked as a translational fusion to a gene encoding a highly abundant secreted protein via a linker.

In the process of producing recombinant bovine myglobin in T. reesei, the gene fusion follows the sequential order of the following motifs: a signal sequence followed by the catalytic domain of the highly abundant secreted protein, which is connected via a flexible neutral polyglycine linker (4×GGGGS, 3×GGGS, 3×Gly or 5×Gly) to an artificial protease cleavage site (e.g. recognized by Kex2 protease, or an AQ/RQ proteolytic cleavage site), and then the codon-optimized bovine myoglobin gene. Kex2 protease recognizes and cleaves dibasic and acid/basic residues on proteins targeted for secretion, such as cleavage site AAAAGA. For example, to enhance recombinant bovine myoglobin yields, Aspergillus niger GlaA can be used as carrier when fused to the codon-optimized bovine myoglobin gene. Other proteins useful as carriers are the Aspergillus niger var. awamori α-amylases AmyA and AmyB, or the T. reesei CBM domain. Among the many engineering strategies, fusing the gene of interest to a co-secretion partner such as cellobiohydrolase I can be performed to ease the secretion and mRNA stabilization.

In the process of producing recombinant bovine myglobin in T. reesei, the gene fusion can also direct the recombinant protein into protein bodies (PB) in the endoplasmic reticulum (ER). For example, gene fusions of hydrophobin 1 (hfb1) coding region connected to the codon-optimized bovine myoglobin gene may allow the formation of agglomerated PBs that can be isolated by altering hydrophobicity of of their fusion partner using an aqueous two-phase system. Another example for PB formation corresponds to using Zein proteins as fusion partners, since thes proteins are imported in the ER and localize to the periphery of the PBs surrounding aggregates. Fusion of the codon-optimized bovine myoglobin gene to synthetic zera peptide gene may protect the heterologous protein from proteolysis and allow purification from fungal PBs.

In some embodiments, the expression vector includes a selectable marker. Examples of selectable markers include those that confer antibiotic resistance. Nutritional markers may also be used in the present invention and include markers well known to those skilled in the art, such as amdS (A. nidulans acetamidase), argB (A. nidulans ornithine carbamoyl transferase) and pyr4 (T. reesei orotidine-5′-monophosphate decarboxylase). In some embodiments, the expression vector is also a replicon, a gene encoding antibiotic resistance to allow selection of bacteria harboring the recombinant plasmid, and/or a plasmid vector for insertion of heterologous sequences. Includes unique restriction sites present in non-critical areas. Any useful antibiotic resistance gene may be used in the present invention. For expression vectors, a prokaryotic sequence is preferably chosen that does not interfere with replication or integration of T. reesei DNA.

Preferred selection markers that confer antibiotic resistance are the Streptomyces hygroscopicum phosphinothricin acetyltransferase or glufosinate/phosphinothricin/biapholos resistance gene (pat/bar), E. coli hygromycin phosphotransferase or hygromycin resistance gene (hph or hpt); Strepotalloteichus hindustanus bleomycin/phleomycin resistance gene (ble), Neurospora crassa β-tubulin gene for benomyl resistance (bml or ben), A. oryzae pyrithiamine resistance (ptrA), Streptomyces noursei nourseothricin acetyltransferase (CaNATI), neomycin phosphotransferase (NPTII), among others known in the art.

Also, selectively removal of the selectable marker genes from the fungal genome can be considered. It can be performed by using site-specific recombinase systems to selectively excise the marker genes from the fungal genome, or the cloning of the selectable marker gene between fungal transposable elements, resulting in the excision of the selectable marker gene. For example, the marker cassette can be looped out of the chromosomal DNA by the action of Cre recombinase. In this case, the cassette includes two loxP sites flanking the selectable marker genes, and a chromosomal gene (e.g. pyr4 gene) is replaced with a cassette containing Cre recombinase under the control of a fungal promoter.

In another embodiment, transformation can be performed using plasmids carrying selectable markers amdS or the argB gene of A. nidulans, which complements the respective argB gene of T. reesei. For example, for adenine auxotrophic strains, the expression cassette can be inserted at the ade2 locus together at the pyr2 gene used as marker. Also, a double-deletion strain can be used to allow multiple genomic manipulation steps. Other auxotrophic markers that can be used are asl1 (encoding an enzyme of the 1-arginine biosynthesis pathway), the hah1 (encoding an enzyme of the 1-lysine biosynthesis pathway), the dominant-nutritional marker suc1 (sucrose utilization), among others. More preferred selection markers are the argB or the amdS genes.

### Fungal Transformation and Cultivation

Fungal transformation can be performed using different techniques, such as polyethylene glycol (PEG)-mediated transformation of protoplasts, particle bombardment, electroporation, and Agrobacterium tumefaciens-mediated transformation.

The transforming cassette can be integrated at several different locations, resulting in multiple tandem copies in the T. reesei genome. Cassette copy number is quantified by q-PCR using a single copy gene as reference (e.g., sar1 gene). Sequences

Insertion sites are analyzed by southern blot using probes containing the 250 bp from the codon-optimized bovine myoglobin gene.

The fungal cells of the invention are cultured in nutrient media suitable for the production of recombinant bovine myoglobin using methods well known in the art. For example, the cells may be used in research or industrial fermentation under conditions in a suitable medium capable of expressing and/or isolating the recombinant bovine myoglobin (continuous, batch fed-batch (fed-batch) or shaking flask culture (including solid fermentation), small or large scale fermentation. Culture is performed using procedures well known in the art.

The fungal cells of the present invention are cultivated in a nutrient medium suitable for production of the recombinant bovine myoglobin using methods known in the art. For example, the cultures are performed in suitable nutrient media containing carbon and nitrogen sources and inorganic salts (eg, Benn ett, J W and Lasure, L., eds., More Gene Manipulations in Fungi, Academic Press. CA, 1991). Preferred media for T. reesei are: PDB (potato-dextrose broth), YPD (20 g/L bacto peptone, 10 g/L yeast extract, 20 g/L D-glucose); YVD (20 g/L vegetable peptone or vegetable tryptone, 10 g/L yeast extract, 20 g/L D-glucose). The preferred nitrogen sources are vegetable protein hydrolizates suitable for microbiology, such as commercial vegetable peptones (e.g., soybean, wheat, peas, potatoes, etc., hydrolyzed with animal-free enzymes, such as papain). Other media used for T. reesei culture are: SCD (6.7 g/L yeast nitrogen base, 20 g/L D-glucose), cellulase repressing medium with glucose or CRM (10 g/L glucose, 20 g/L yeast extract, 37.8 mM (NH₄)₂SO₄, 36.7 mM KH₂PO₄, 2.4 mM MgSO₄, 4.1 mM CaCl₂, 15.6 M CoCl₂, 18.0 M FeSO₄, 4.9 M ZnSO₄ and 9.5 M MnSO₄), and Trichoderma minimal medium or TMM (5 g/L (NH₄)₂SO₄, 15 g/L KH₂PO₄, 0.6 g/L MgSO₄, 0.6 g/L CaCl₂, 0.2 mg/L CoCl₂, 5 mg/L FeSO₄, 1.4 mg/L ZnSO₄ and 1.6 mg/L MnSO₄), where the carbon source is 20 g/L glucose or 20 g/L glycerol. In case of agar plate cultivations, 20 g/L agar is added to liquid medium in addition to the components mentioned above.

For carbon source replacement experiments, mycelia are precultured in 250 mL of Mandels-Andreotti (MA) medium supplemented with 10 g/L glucose as the sole carbon source at 180 rpm at 30° C. for 24 h. Pregrown mycelia is washed, and equal amounts are resuspended in 20 mL MA medium containing 10 g/L glucose and incubated for 8 h at 30° C. For protein studies, strains are cultured in MA medium without peptone and supplemented with 20 g/L glucose, 20 g/L fructose or 20 g/L glycerol as the sole carbon sources. Cultures are inoculated with 10⁶ conidia/L.

T. reesei is cultured at temperatures of about 30° C. (e.g., 25-35° C.), or at a temperature of about 37° C. (e.g., 34-39° C.), for example.

The types and abundance of secreted and intracellular proteins in T. reesei are strongly related to the carbon source and the light exposition. The growth medium may contain cellulose, glucose, lactose, sophorose, glycerol, cellobiose, and/or other sugar or cellulose-related materials. Also, to enhance protein secretion in T. reesei, the fungal cells are grown under different light conditions, ranging from 700 to 5,000 lux. Transformed strains can be grown either in the presence of constant illumination (day light simulating wave length distribution) with different light intensities ranging from 700 to 5,000 lux or in constant darkness.

In some embodiments, the method for producing recombinant bovine myoglobin in T. reesei involves culturing fungal host cells under batch or continuous fermentation conditions. The batch fermentation occurs in a closed system, wherein the composition of the medium is set at the beginning of the fermentation with no addition of any components to the system, and the medium is inoculated at the beginning of the fermentation. Within batch cultures, cells progress through a static lag phase to a high growth log phase and to a stationary phase where growth rate is diminished or halted. If untreated, cells in the stationary phase eventually die.

In another embodiment, the method for producing recombinant bovine myoglobin in T. reesei involves culturing fungal host cells in a “fed-batch fermentation” system. In this system, the substrate is added in increments as the fermentation progresses. Fed-batch systems are useful when catabolite repression inhibits the metabolism of the cells and where it is desirable to have limited amounts of substrate in the medium. The substrate concentration in fed-batch systems is usually is estimated on the basis of the changes of measurable factors such as pH, dissolved oxygen and the partial pressure of waste gases such as CO2.

In other embodiments, the method for producing recombinant bovine myoglobin in T. reesei involves culturing fungal host cells in continuous fermentation. This is an open system where a defined fermentation medium is added continuously to a bioreactor and an equal amount of medium is simultaneously removed for processing. The fungal cultures are maintained at a constant high density (log phase growth). In continuous fermentation, cell loss due to medium removal must be balanced against the cell growth rate in the fermentation. Methods of modulating nutrients and growth factors for continuous fermentation processes are well known in the art.

In another embodiment, the method for producing recombinant bovine myoglobin in T. reesei involves culturing fungal host cells on a selected solid support, such as food industrial residues (e.g., rice, wheat bran, fruit and potato peels) or algal wastes. In solid state fermentation (SSF), the fungal host mycelia spread onto the surface of solid materials, where aerobic fermentation occurs. The use of SSF for production of recombinant bovine myoglobin in T. reesei allows the reduction of energy and water consumption, and the fermented residue can be used as single cell protein or as substrate for production of hydrolyzed protein for feeds.

### DNA Constructions

E. coli DH5a is used as the host strain for the recombinant DNA manipulations. Bacterial cultivations were performed in Luria-Bertani broth with ampicillin (10 g/L tryptone, 5 g/L yeast extract, 0.17 M NaCl and 100 g/mL ampicillin).

DNA constructs in the present invention are performed using techniques known in the art. In some embodiments, genes cloning is performed using restriction sites, in vitro assembly reactions (Gibson assembly, Golden Gate assembly), and/or in vivo assembly reactions (Saccharomyces-mediated DNA assembly).

T. reesei genome editing is performed using the CRISPR/Cas9 system. See, C. S. Nødvig, et al., “A CRISPR-Cas9 System for Genetic Engineering of Filamentous Fungi,” PLOS One, 2015, Vol. 10, Issue 7 e0133085. doi:10.1371/journal.pone.0133085, the entire contents of which are hereby incorporated by reference in their entirety.

In the process of preparing recombinant bovine myoglobin in T. reesei, the in vitro coupling of separately manufactured myoglobin and heme B, T. reesei is used as a production host of bovine myoglobin, and a second microorganism (e.g. yeast) is used as a production host of heme.

### Recombinant Bovine Myoglobin Recovery

Recombinant bovine myoglobin from T. reesei can be recovered directly from the medium by methods well known in the art. For example, recombinant bovine myoglobin can be recovered from feeding media by conventional procedures including, but not limited to, centrifugation, filtration, ultrafiltration, extraction, spray-drying, freeze-drying, evaporation, or precipitation. Next, the recovered protein can be further purified by various chromatography methods, for example, ion exchange chromatography, gel filtration chromatography, affinity chromatography and the like.

### SUMMARY OF THE INVENTION

The present invention describes a method to express myoglobin in T. reesei. In an aspect, a method for preparing a bovine myoglobin includes: constructing a plasmid containing a codon-optimized gene for bovine myoglobin; constructing a T. reesei production host containing the expression plasmid; producing the recombinant bovine myoglobin by culturing the first T. reesei production host; and collecting the myoglobin from a feeding media. In another aspect, the heme biosynthesis can be enhanced by the co-expression of porphyrin pathway enzymes, such as ALAS, ALAD, PBGD, UROD, and/or a ferrochelatase.

The recombinant bovine myoglobin of the present invention can be formulated as food compositions that include all forms, such as functional foods, nutritional supplements, health foods and food additives. The food compositions of the above types can be produced in various forms by conventional methods known in the art. For example, in liquid form, granules, encapsulated or powdered. In addition, the recombinant bovine myoglobin of the present invention can be mixed with known substances or active ingredients known to have an effect of reducing advanced oxidation end products to produce a composition. Functional foods include vegetables and processed foods thereof (e.g., canned vegetables, bottled foods, macaroni, etc.), and plant-based processed foods thereof (for example, plant-based ham, sausage, hamburgers), breads and noodles (e.g., spaghetti, macaroni, ramen, etc.), edible vegetable fats, vegetable oils, and seasonings (e.g. miso, soy sauce, sauces, etc.).

The recombinant bovine myoglobin of the present invention can be formulated using techniques known in the art for liquids, powders, granules, capsules, gels, syrups, slurrys, suspensions, concentrated solutions and the like. For example, powdered and granular formulations can be prepared blending the recombinant bovine myoglobin with a solid excipient, grinding it, adding a suitable auxiliary agent, and then processing it into a granular mixture. Examples of suitable excipients include sugars including lactose, sorbitol, mannitol, xylitol, erythritol and maltitol, starches including corn starch, wheat starch, rice starch and potato starch, cellulose and the like. Methyl cellulose, including sodium carboxymethyl cellulose and hydroxypropyl methyl-cellulose, and the like can be included. In some cases, the food compositions of the present invention may further include antioxidants, lubricants, wetting agents, emulsifiers and preservatives.

The recombinant bovine myoglobin of the present invention can be used as an agent to increase proliferation and metabolic activity of mammalian muscle satellite cells (e.g., bovine, porcine, poultry, etc.) in cultured-meat. Also, the recombinant bovine myoglobin can be used as an agent for modifying the cultured-meat color to resemble to beef.

The use of the recombinant bovine myoglobin of the present invention is not limited to this, but is preferably in the finally produced food in 0.01 to 99.0% by weight.

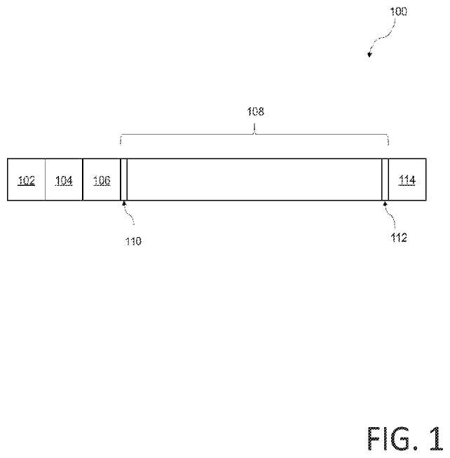

Specifically, FIG. 1 depicts a block diagram of a codon-optimized bovine myoglobin sequence 100 used in this technology. The codon-optimized bovine myoglobin sequence 100 includes a PacI gene or site 102, a Kozak sequence 104, a signal sequence 106, the codon-optimized myoglobin opening reading frame (ORF) 108, and an Xbal restrictive enzyme or site 114.

As described herein, the PacI gene or site 102 encodes a product similar in structure to eukaryotic Ca(²⁺)-ATPases. As described herein, the signal peptide 106 is a short peptide present at the N-terminus of most newly synthesized proteins, carrying information for protein secretion. As described herein, the XbaI restrictive enzyme or site 114 recognizes T\hat{ }CTAGA sites.

As described herein, the Kozak sequence 104 is a nucleic acid motif that functions as the protein translation initiation site in most eukaryotic mRNA transcripts. Regarded as the optimum sequence for initiating translation in eukaryotes, the sequence is an integral aspect of protein regulation and overall cellular health as well as having implications in human disease.

It should be appreciated that, as shown in FIG. 1, the myoglobin ORF 108 includes an ATG codon 110 and a Stop codon 112. The myoglobin ORF 108 is a Bos taurus (e.g., a European cattle) codon-optimized to a Trichoderma reesei host.

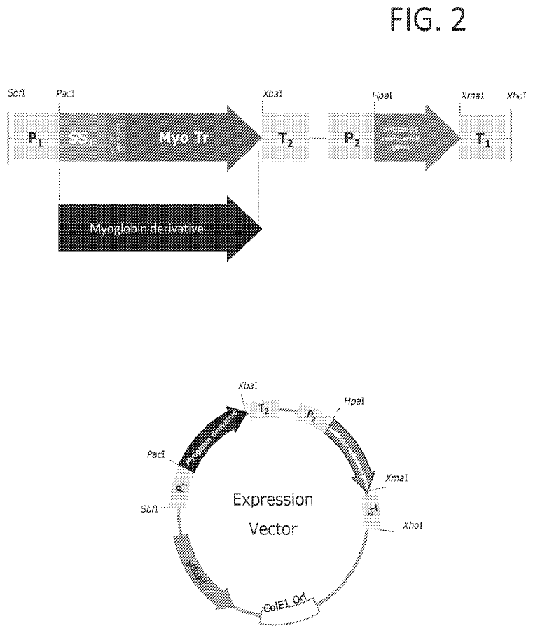

The FIG. 2 shows a schematic representation of a plasmid for recombinant expression of a myoglobin derivative in Trichoderma reesei. In accordance with embodiments of the present invention of the construct comprising the following features: SbfI restriction site; first promoter (P₁); PacI restriction site; a secretion signal sequence (SS₁); a linker; a Kexin site (e.g. an AQ/RQ proteolytic cleavage site); a myoglobin derivative gene codon-optimized to a Trichoderma reesei host; XbaI restriction site; first terminator (T₁); second promoter (P₂); HpaI restriction site; antibiotic resistance gene; XmaI restriction site; second terminator (T₂); XhoI restriction site.

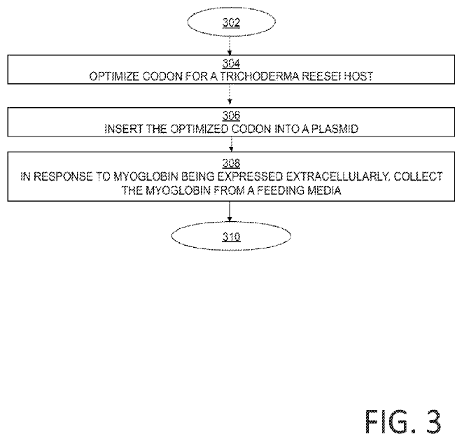

The method to express myoglobin in T. reesei and collect the myoglobin from a feeding media may include numerous process steps, as shown in FIG. 3. The method of FIG. 3 begins at a process step 302, which is followed by a process step 304 that includes optimizing a codon for a T. reesei host. Software, such as GenScript, IDT-DNA or EUGENE (Universidade de Aveiro, Portugal) may be used to optimize the codon for the T. reesei host. The codon optimization for expression in T. reesei can also be based on the complete codon usage of T. reesei deposited on Kazusa DNA Research Institute (No 51453) and using the tool from the Evolutionary Genomics Group, Universitat Rovira i Virgili (URV, Tarragona. Spain.) Also, the codon-optimized bovine myoglobin gene is flanked with PacI and XbaI restriction sites to allow their subsequent cloning into the plasmid pTrEno, which is used as parent vector for construction of the expression vectors used in this invention, pTrPdc-MyoTr and pTrGdp-MyoTr. All the coding genes designed, optimized for codon usage of T. reesei, and chemically synthesized devoid of XhoI, SbfI, HindIII, EcoRI, SacI, PacI, XbaI and SacII restriction sites to allow subcloning in the parent vector pTrEno, and pTrPdc-MyoTr and pTrGdp-MyoTr, including their derivatives or other expression plasmids, Table 1 below depicts a number of different base pairs between the bovine wild-type sequence and the optimized codon sequence for T. reesei.

TABLE 1

| | Bovine | Optimized Codon |
|---|---|---|
| | Wild-Type | Sequence |
| | Sequence | for T. reesei |
| Bovine Wild-Type Sequence | | 74 |
| Optimized Codon Sequence | 74 | |
| for T. reesei | | |

A process step 306 follows the process step 304 and includes inserting the optimized codon into a plasmid. In preferred examples, the plasmid comprises a pTrEno plasmid having a constructive promoter. It should be appreciated that the purpose of the pTrEno plasmid is as an enolase-promoter driven expression vector for protein production in T. reesei. See, J. G. Linger, et al., “A Constitutive Expression System for Glycosyl Hydrolase Family 7 Cellobiohydrolases in Hypocrea jecorina,” Biotechnol Biofuels., 2015, Vol. 8, Issue 45, doi: 10.1186/s13068-015-0230-2, eCollection 2015, the entire contents of which are hereby incorporated by reference in their entirety. The plasmid for bovine myoglobin expression in T. reesei is shown in FIG. 2.

A process step 308 of the method of FIG. 3 follows the process step 306 and includes collecting the myoglobin from a feeding media in response to the myoglobin being expressed extracellularly. A process step 310 follows the process step 308 to conclude the method of FIG. 3. As an optional step following the process step 308, the method may also include: purifying the myoglobin. As described, the collected myoglobin may be used in plant-based food products to mimic flavors associated with cooked ground meat.

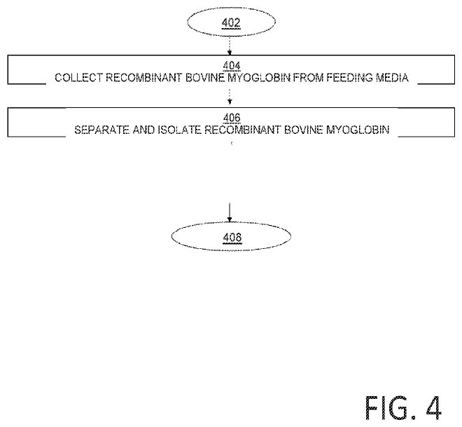

A process step 402 of the method of FIG. 4 follows the process step 404 and includes collecting the myoglobin from a feeding media in response to the myoglobin being expressed extracellularly. A process step 406 of separating and isolating recombinant bovine myoglobin follows the process step 408 to conclude the method of FIG. 4. As an optional step following the process step 406, the method may also include: purifying the myoglobin. As described, the collected myoglobin may be used in plant-based food products to mimic flavors associated with cooked ground meat.

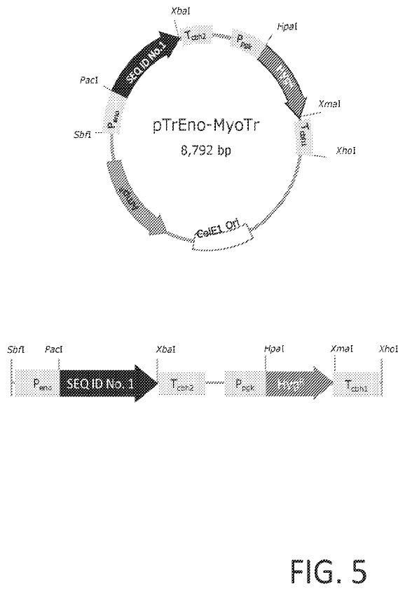

FIG. 5 shows a schematic representation of the parent vector pTrEno-MyoTr used for subcloning and screening of protein expression. This plasmid comprises the following features: SbfI restriction site; promoter eno (Pₑₙₒ); PacI restriction site; SEQ ID NO: 1: Kozak sequence, a cbh1 signal sequence; and Bos taurus myoglobin codon-optimized to a Trichoderma reesei host; XbaI restriction site; cbh2 terminator; promoter pgk (P_{pgk}); HpaI restriction site; hygromycin resistance gene; XmaI restriction site; cbh1 terminator; XhoI restriction site.

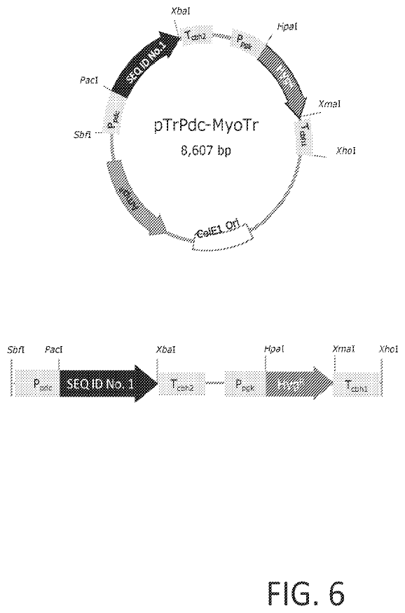

FIG. 6 shows a schematic representation of the expression vector pTrPdc-MyoTr used for myoglobin expression that comprise the following features: SbfI restriction site; pdc promoter (P_{pdc}); PacI restriction site; SEQ ID No. 1: Kozak sequence, a cbh1 signal sequence; and Bos taurus myoglobin codon-optimized to a Trichoderma reesei host; XbaI restriction site; cbh2 terminator; promoter pgk (P_{pgk}); HpaI restriction site; hygromycin resistance gene; XmaI restriction site; cbh1 terminator; XhoI restriction site.

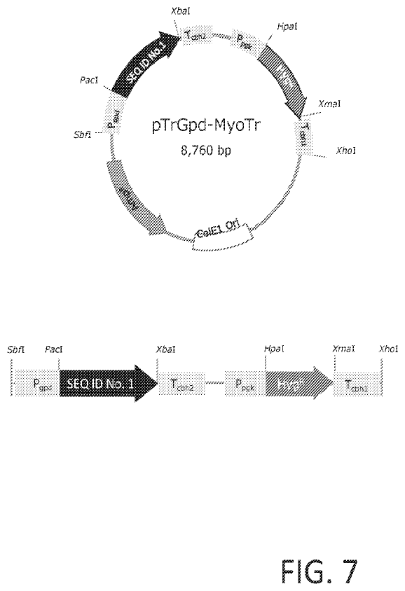

FIG. 7 shows a schematic representation of the expression vector pTrGpd-MyoTr used for myoglobin expression and comprises the following features: SbfI restriction site; gpd promoter (P_{gpd}); PacI restriction site; SEQ ID NO: 1: Kozak sequence, a cbh1 signal sequence; and Bos taurus myoglobin codon-optimized to a Trichoderma reesei host; XbaI restriction site; cbh2 terminator; promoter pgk (P_{pgk}); HpaI restriction site; hygromycin resistance gene; XmaI restriction site; cbh1 terminator; XhoI restriction site.

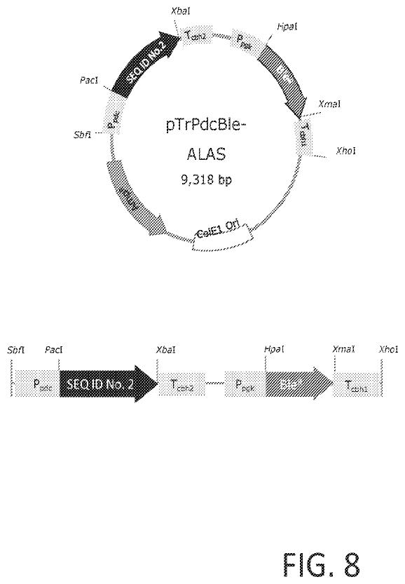

FIG. 8 shows a schematic representation of the expression vector pTrPdcBle-ALAS used for expression of 5-aminolevunic acid synthase (ALAS) and comprises the following features: SbfI restriction site; pdc promoter (P_{pdc}); PacI restriction site; SEQ ID NO: 3: Kozak sequence and Trichoderma reesei ALAS synthase gene; XbaI restriction site; cbh2 terminator; promoter pgk (P_{pgk}); HpaI restriction site; bleomycin/phleomycin resistance gene; XmaI restriction site; cbh1 terminator; XhoI restriction site.

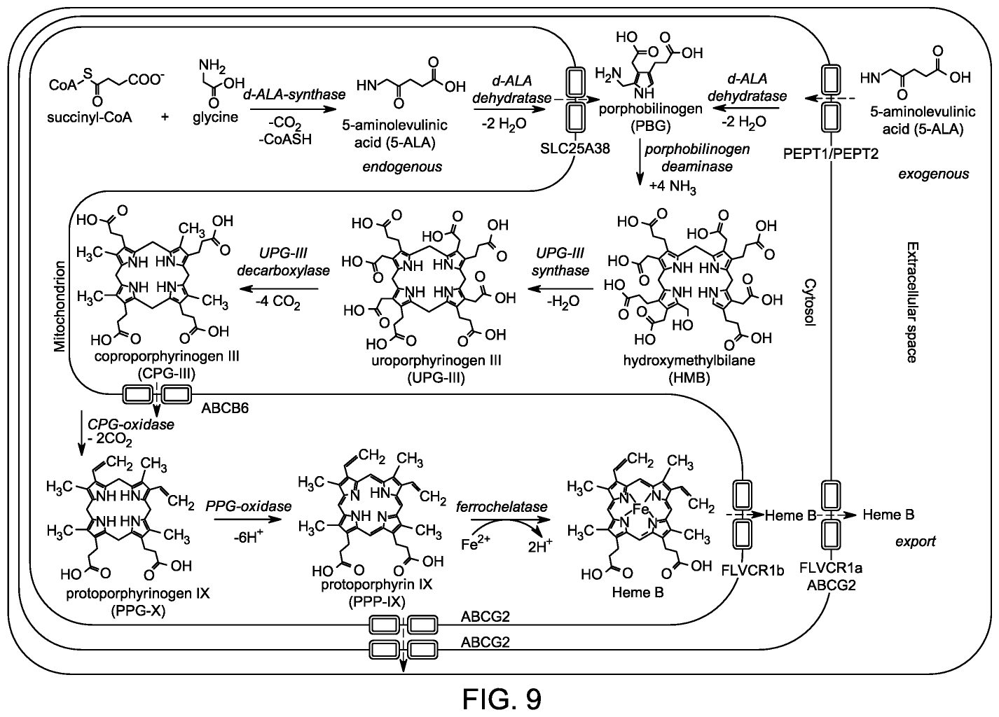

FIG. 9 depicts the porphyrin synthesis pathway.

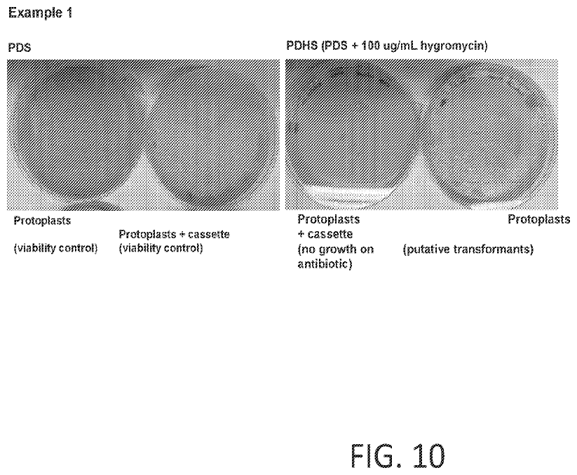

FIG. 10 shows T. reesei transformation plates.

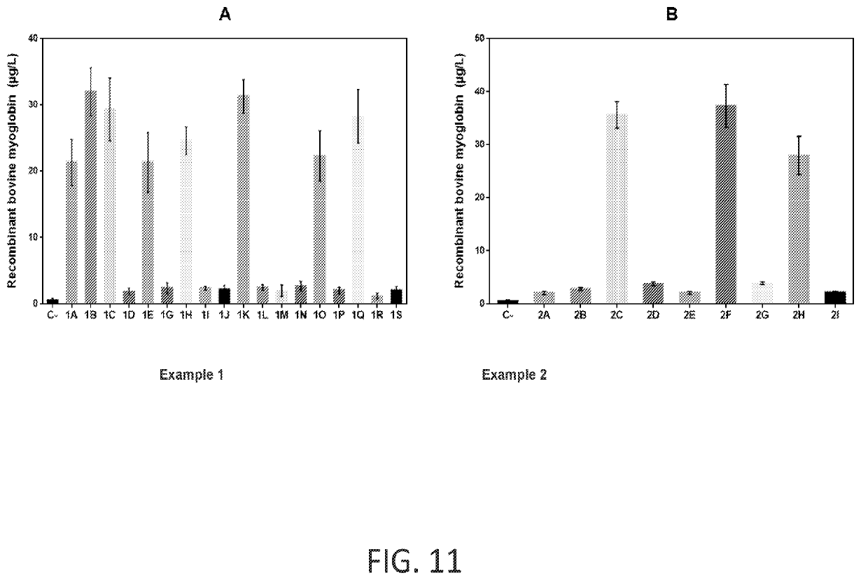

FIG. 11 shows recombinant bovine myoglobin yields in culture supernatants from T. reesei transformed with pTrEno-MyoTr cassette, as described in Example 1 (FIG. 11-A) and Example 2 (FIG. 11-B).

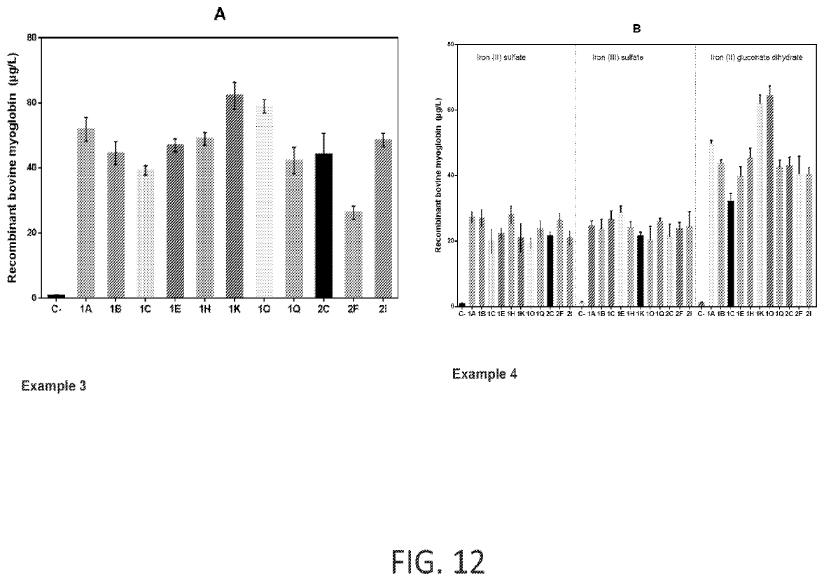

FIG. 12 shows the effects of exogenous iron in recombinant bovine myoglobin yields. T. reesei transformed with pTrEno-MyoTr was cultured supplemented with exogenous heme-iron, as described in Example 3 (12-A); and with non heme-iron, as described in Example 4 (12-B).

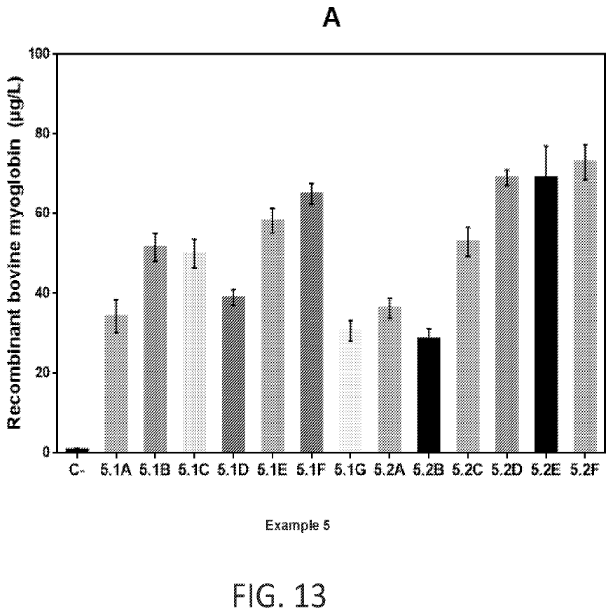

FIG. 13 shows recombinant bovine myoglobin yields in culture supernatants from T. reesei transformed with pTrPdc-MyoTr (strains 5.1A-5.1G) and pTrGdp-MyoTr (strains 5.2A-5.2F), as described in Example 5.

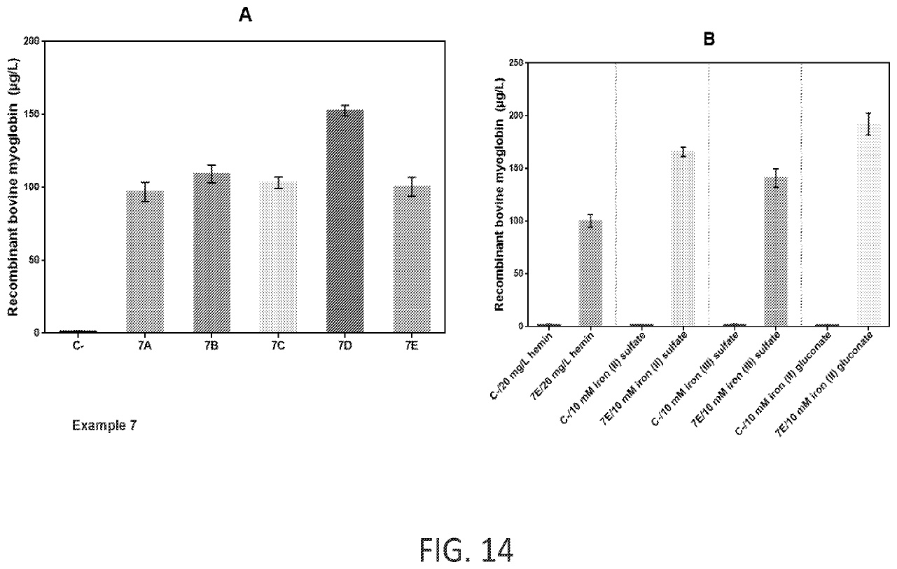

FIG. 14 shows recombinant bovine myoglobin yields in culture supernatants from T. reesei co expressing recombinant bovine myogolobin and porphyrin synthesis enzyme ALAS (fungal host cotransformed with pTrGdp-MyoTr and pTrPdcBle-ALAS, FIG. 14-A), as described in Example 7. FIG. 14-B shows the effects of iron supplementation (heme and non heme) in myoglobin yields in a selected strain (7E), as described in Example 8.

### Production of Active Myoglobin

The conversion of an apoprotein into a hemoprotein is related to the availability of heme provided by the heme biosynthetic pathway. The apoprotein form of the hemoprotein combines with heme to produce the active hemoprotein. The resulting hemoprotein is more stable against proteolytic attack than the apoprotein. When the production rate of the apoprotein is higher than that of heme produced, the apoprotein will accumulate and undergo proteolytic degradation lowering the yield of the active hemoprotein. Therefore, for production of active recombinant bovine myoglobin, the prosthetic group heme B should be available during fermentation.

To enhance the availability of heme B for the successful expression of active recombinant bovine myoglobin in T. reesei there are many strategies, as follows: culture supplementation with iron and iron-related compounds; engineering the fungal porphyrin synthesis pathway to increase heme B; the overexpression, deregulation and/or deletion of heme-transport or heme-exporter genes; and/or the overexpression, deregulation and/or deletion of genes and transcription factors involved in oxygen sensing pathway; among others.

### Enhancement of Heme B Availability Via Culture Supplementation

In animal foods, 40% of the total iron content is heme-iron and the rest 60% of it is non heme-iron. Iron present in vegetable foods is all non heme-iron. The most common heme-iron sources are hemoglobin and myoglobin from mammalian blood and muscle tissues. These heme-proteins contain heme B. The bioavailability of heme-iron is known to be much higher than that of non-heme iron, and the absorption of heme-iron in the body is not affected by other dietary factors. Therefore, heme-iron production in microbial cells is advantageous, whether it is used as food supplement for mammals or as microbial culture supplement for the heterologous expression of heme-proteins. The most common sources of non heme-iron sources are inorganic iron compounds, such as iron (II) chloride, iron (II) sulfate, iron (III) sulfate, iron (II) citrate, iron (II) lactate, iron (II) glucuronate, among others.

To enhance heme B availability for producing recombinant bovine myoglobin in T. reesei, the culture media can be supplemented with external heme B or hemin, a heme-iron compound sourced from animal blood cells. Hemin can be added to the microbial culture media between 0.1 to 100 mg/L, thus the recombinant cells expressing bovine myoglobin capture hemin and attach it to the recombinant bovine myoglobin during the whole production process. However, hemin is an expensive animal-based by-product and it is not the preferred supplement for producing recombinant bovine myoglobin in a complete animal-free process. In one embodiment of the present invention, hemin is added into the microbial culture media between 10 to 100 mg/L to enhance the heme availability when culturing T. reesei recombinant strains expressing bovine myoglobin and increase bovine myoglobin yields.

Another strategy to enhance heme B availability for recombinant bovine myoglobin expression in T. reesei is supplementing the culture media using inorganic and/or organic iron compounds, such as iron (II) sulfate, iron (III) sulfate, iron (II) chloride, iron (II) D-glucuronate, iron (II) gluconate, iron (II) fumarate, iron (II) citrate, iron (II) lactate, their solvates or hydrates, among other iron compounds. Using non heme-iron compounds in the culture media is a good alternative in animal free/based processes and allows the iron coordination into the porphyrin ring precursor (protoporphyrin IX or PPP-IX) endogenously produced by the microbial cell. Therefore, in one embodiment of the present invention, the heme availability is enhanced supplementing culture media with non heme-iron compounds in the range between 10 μM to 10 mM of ferrous ion, increasing the yields of recombinant bovine myoglobin expressed by T. reesei.

Another strategy to increase the heme levels is supplementing the culture media with limiting intermediate compounds involved in the porphyrin synthesis pathway. For example, adding the precursor 5-aminolevulinic acid (5-ALA), prior to or throughout the fermentation.

Therefore, in one embodiment of the present invention, the heme availability in T. reesei recombinant strains expressing bovine myoglobin is enhanced using 5-ALA in the culture media in the range between 1 μM to 10 mM.

### Enhancement of Endogenous Heme Production Via Engineering Porphyrin Synthesis Pathway

Another strategy to produce the active form of recombinant bovine myoglobin in T. reesei involves balancing the expression of recombinant bovine myoglobin with an enhanced endogenous heme production. This strategy can be performed overexpressing enzymes involved in the fungal biosynthesis pathway of heme-iron or porphyrin synthesis pathway.

The biosynthesis pathway of heme-iron has been well studied in many vertebrates and microbial species. Heme-iron is produced in eukaryotes, such as animals, plants, fungi and insects, through the porphyrin biosynthesis pathway via 5-aminolevulinic acid (5-ALA or d-ALA) from the citric acid cycle. In these organisms, eight 5-ALA molecules result from the reaction of the amino acid glycine with succinyl-CoA in the mitochondria, which is catalyzed by Δ-aminolevulinic acid synthase (Δ-ALAS or ALAS). The porphyrin biosynthesis starts from two 5-ALA molecules that are then combined by ALA-dehydratase (ALAD) to give porphobilinogen (PBG), which contains a single pyrrole ring. Zinc is essential for ALAD activity. The 5-ALA molecules can enter from the extracellular space to the cytosol, where ALAD converts 5-ALA to PBG. Four PBGs are then combined through deamination into hydroxymethylbilane (HMB) by porphobilinogen deaminase (PBGD), releasing four ammonia molecules. HMB is hydrolyzed to form the circular tetrapyrrole uroporphyrinogen III (UPGIII) by uroporphyrinogen III synthase (UROS), resulting each pyrrole ring with hydrogen atoms on its two outermost carbons replaced by an acetic acid group (—CH₂—COOH, “A”) and a propionic acid group (—CH₂—CH₂—COOH, “P”). UPGIII is then converted into coproporphyrinogen III (CPGIII) by the enzyme uroporphyrinogen III decarboxylase (UPG-decarboxylase or UROD). Then, CPGIII is transported into the mitochondria, where CPG-oxidase (CPO) converts CPG-III into protoporphyrinogen IX (PPGIX). Then, PPGIX is oxidized by PPG oxidase (PPO) to give the main end-product, protoporphyrin IX (PPIX), which is combined with iron by ferrochelatase (FC) to form heme B. Therefore, 5-ALA is a crucial substrate for heme-iron biosynthesis.

The present inventors have studied to design a method for biologically producing heme-iron by increasing the intracellular production of the enzymes involved in the porphyrin synthesis pathway, overcoming the problems of using animal blood-based heme-iron products.

One embodiment for the method for biologically producing heme-iron involves increasing intracellular 5-ALA by coexpressing ALAS, ALAD, PBGD, UROD, and/or FC. The coexpression of these enzymes is carried out using a vector with a selectable marker different to the one contained in the expression vector for bovine myoglobin.

Enhancement of Endogenous Heme Production Via Engineering Oxygen Sensing Pathway Another strategy involves altering the fungal oxygen sensing pathway. It can be performed overexpressing, derepressing, repressing or deleting genes related to the general oxidative stress response, and/or transcription factors involved in the oxygen sensing pathway, also by activating hypoxia-induced genes. The expression of the majority of the genes involved in the heme synthesis is largely constitutive and not affected by oxygen, with the exception of CPG-oxidase in the mitochondria. In S. cerevisiae, intracellular heme synthesis is directly correlated with the environmental oxygen availability. In this yeast, heme production is regulated through control of CPG-oxidase endogenous expression by Hap1p gene and Rox1p transcriptional repressor, because the products of HAP1 and ROX1 genes repress CPG-oxidase expression in the presence of high heme levels. HAP1 regulates the transcription of many genes involved in yeast cell response to oxygen availability. It has been described that the deletion of HAP1 in yeasts resulted in enhanced endogenous heme production when overexpressing heme-proteins. See Martinez J. L. et al., “Engineering the Oxygen Sensing Regulation Results in an Enhanced Recombinant Human Hemoglobin Production by Saccharomyces cerevisiae,” Biotechnology and Bioengineering, January 2015, Volume 112, Pages 181-188, the entire contents of which are hereby incorporated by reference in their entirety.

In one embodiment of the present invention, the heme B availability is enhanced by deletion or truncation of HAP1 gene homolog of T. reesei in the T. reesei recombinant strains expressing bovine myoglobin.

Heme availability enhancement can also be reached by increasing heme-trafficking and membrane transport due to overexpression of mitochondrion heme-membrane transporters. For example, Flvcr1b is a mitochondrial heme-transporter that is present in mammals and promotes heme efflux into the cytoplasm. On the other hand, the mitochondrial coproporphyrin transporter ABCB6 (homolog to ATM1 gene in S. cerevisiae) up regulates de novo porphyrin synthesis increasing CPGIII in the mitochondria. The overexpression of ATM1 product in yeasts increases the expression of several rate-limiting enzymes (CPO, ALAD, and ALAS) in the heme biosynthetic pathway of yeasts, resulting in higher heme levels in the cytosol.

In one embodiment, heme B amount is enhanced by co expressing a T. reesei homolog of Flvcr1b transporter in the T. reesei recombinant strain that expresses bovine myoglobin.

In another embodiment, heme B amount is enhanced by co expressing a T. reesei homolog of ABCB6 transporter in the T. reesei recombinant strain that expresses bovine myoglobin.

In Trichoderma genus, the heme B group is used in the biosynthesis of heme-derived cofactors involved in energy metabolism. Heme B is used as a cytochrome c (Cyt c) cofactor and as precursor of heme A, the cytochrome c oxidase (Cyc c oxidase) cofactor. Cyt c is a mitochondrial intermembrane water-soluble heme-protein and it is a critical component of the respiratory electron transport chain because is responsible of transferring electrons between Coenzyme Q-Cyt c reductase (Complex III) and Cyt c oxidase (Complex IV). Also, Cyt c triggers apoptosis when released into the cytosol. On the other hand, Cyt c oxidase (Complex IV) is a hydrophobic transmembrane protein and acts as the last enzyme in the respiratory electron transport chain of cells.

As Cyt c cofactor, heme B is linked to Cyt c by a cytochrome-c heme-lyase (E.C. 4.4.1.17). On the other hand, heme B is used for the production of heme A, the Cyt c oxidase cofactor. In this route, heme B is firstly modified into heme O by a protoheme farnesyltransferase-like protein (EC: 2.5.1.141) adding a hydroxyethylfarnesyl moiety at the ring's position 2. Then, heme O is modified into heme A by an oxidoreductase (E.C. 1.17.99.9) that oxidizes the methyl group into a formyl moiety in the ring's position 8. Although T. reesei is an obligatory aerobic organism,

In some embodiments, endogenous heme B availability is enhanced in a recombinant T. reesei strain expressing bovine myoglobin by the deletion or truncation of Cyt c or Cyt c oxidase genes, and/or the deletion or repression of genes involved in Cyt c oxidase assembly.

### EXAMPLES

### Example 1

The expression cassettes harboring codon-optimized bovine myoglobin gene were cloned into the pTrEno plasmid using cloning sites PacI and XbaI. For fungal transformation, the expression cassette was released from pTrEno by restriction digestion with SbfI and XhoI (New England Biolabs), separated on a 1% agarose gel, and purified with GeneJet Gel Extraction kit (ThermoFisher, USA).

Transformation of T. reesei QM6a was carried out as follows. Spores (5×10⁶) of the fungal host strain were plated onto sterile cellophane on potato-dextrose agar and were incubated for 20 hours at 25° C. to allow spore germination and mycelial growth. The cellophane discs with mycelia were transferred to 10 mL of a protoplasting solution containing 0.1 U/mL of lysing enzymes from T. harzianum (Sigma-Aldrich, USA) in 1.2 M sorbitol, 50 mM maleic buffer, pH 5.6. The mycelial mat was digested for 5 hours with shaking at 60 rpm. Protoplasts were separated from undigested mycelia by filtration through sterile No. 30 Miracloth™ and collected into a sterile 50 mL round-bottom centrifuge tube and recovered by centrifugation at 1500×g for 10 min at room temperature. Protoplasts were washed twice with 5 mL of STC buffer (1.2 M sorbitol, 50 mM CaCl₂), 10 mM Tris-HCl, pH 7.5) and centrifuged again at 1500×g for 10 min at room temperature. Protoplasts were resuspended in 250 μL of STC buffer and were incubated with 3-5 μg of linear plasmid fragment (containing the insertion cassette), and 25 μL of PEG solution (40% PEG 6000, 1.2 M sorbitol, 50 mM CaCl₂), 10 mM Tris-HCl, pH 7.5) to assist DNA uptake into the protoplasts. After incubation on ice for 20 min, the transformation mix was diluted with 2 mL of STC buffer and the entire mix was added to 50 mL of molten PDHS agar (potato-dextrose agar containing 1.2 M sorbitol and 100 m/mL hygromycin (shown in FIG. 10). After 4-5 days colonies were transferred to PDA plates without sorbitol for sporulation. Conidia were usually obtained after 3-4 days and purified on PDA plates containing 100 m/mL hygromycin, the colony restrictor Triton X-100 (0.1% v/v) and peptone (0.1% w/v) which accelerates germination. Eighteen putative transformants were obtained after transformation (shown in FIG. 10). After 3 days, single colonies were picked and transferred to minimal medium to allow sporulation and inoculate the microcultures. Sporulation was necessary for the uniform inoculation of cultures. Individual putative transformants that were stable for at least three generations were maintained as spore suspensions at −80° C. in 20% (v/v) glycerol.

For comparison of bovine myoglobin production among all the putative strains, spores from putative strains were cultured in 125-mL flasks containing 20 mL of feeding medium (MA medium with 10 g/L glucose) and fungi were incubated for 5 days at 30° C. at 150 rpm. The relative abundance of the secreted recombinant bovine myoglobin in the feeding medium was measured by ELISA (Abcam Human Myoglobin ELISA Kit, ab171580). Assays were performed in triplicates. Results from ELISA showed that only eight putative transformants expressed and secreted above 20 μg/L of recombinant bovine myoglobin. Untransformed strain (C-) was used as a negative control. Less than 2 μg/L of recombinant bovine myoglobin was detected in the feeding medium of the other ten strains (shown in FIG. 11-A).

T. reesei genomic DNA was extracted using Qiagen DNeasy PowerLyzer Microbial Kit. The insertion of expression cassettes was assessed by PCR using SEQF (SEQ ID NO: 19) and SEQR (SEQ ID NO: 20) primers for sequence analysis (5′ ATGTATCGGAAGTTGGCCGTC and 3′ GACCTGCGACAGACAACCAA). PCR was carried out using Phusion High-Fidelity DNA Polymerase Master Mix (NEB), using 100 ng of genomic DNA as template, and 10 mM of each primer. PCR conditions were as follows: initial denaturation at 98° C. for 30 s; 30 cycles of denaturation at 98° C. for 10 s, annealing at 60° C. for 30 s, extension at 72° C. for 90 s; and final extension at 72° C. for 10 min. Fifteen out of 18 strains were positive for PCR. The eight strains expressing recombinant bovine myoglobin were positive for cassette insertion.

### Example 2

To generate T. reesei QM6a strains expressing bovine myoglobin, vector pTrEno-MyoTr was transformed into QM6a by biolistic gold particle bombardment using PDS-1000/He system (BioRad; E.I. DuPont de Nemours and Company). The vector was prepared as described in Example 1. Gold particles (median diameter of 0.6 μm, BioRad Cat. No. 1652262) were used as microcarriers. The following parameters were used for the transformation: a rupture pressure of 1300 psi, a helium pressure of 29 mm Hg, a gap distance of 0.6 cm, a macrocarrier travel distance of 16 mm, and a target distance of 6 cm. The spore suspension was prepared by washing T. reesei spores from the PDA plates incubated for 4-5 days at 30° C. with sterile deionized water. About 1×10⁷ washed spores were plated on 60 mm diameter plates containing PDA containing 100 μg/mL hygromycin. After particle delivery, the transformation plates were incubated at 30° C. for 5-10 days. After 3 days, the putative strains were transferred to PDA plates containing 100 μg/mL hygromycin. Thirty-four putative transformants were collected and grown in selective PDA plates for at least three generations.

The relative abundance quantitation of the secreted recombinant bovine myoglobin in the feeding medium and the PCR analysis of the putative transformants were measured as described in Example 1. ELISA detected above 20 μg/L myoglobin in the culture supernatants of three putative transformants. Less than 2 μg/L of recombinant bovine myoglobin was detected in the culture supernatants of the other 31 strains. Untransformed strain (C-) was used as a negative control. A selection of ten transformants including the highest myoglobin producers is shown in FIG. 11-B. Three strains expressing and secreting recombinant myoglobin to the feeding medium were positive for PCR using the method described in Example 1.

### Example 3

The effects of heme-iron supplementation in submerged fermentation of recombinant strains from Examples 1 and 2 (total 11 strains) was assessed as follows: spores were cultured in 125-mL flasks containing 20 mL of MA with 10 g/L glucose supplemented with 20 mg/L of hemin. Fungal transformants were incubated for 5 days at 30° C. at 150 rpm. The relative abundance of recombinant bovine myoglobin was measured as described in Example 1. ELISA detected that 9 out of 10 transformants expressed and secreted into the feeding medium more than 40 μg/L of recombinant bovine myoglobin (shown in FIG. 12-A). Untransformed strain (C-) was used as a negative control.

### Example 4

The effects of different non heme-iron supplementation in submerged fermentation of the eleven recombinant strains from Examples 1 and 2 were assessed as follows. Spores were cultured in 125-mL flasks containing 20 mL of feeding medium (MA with 10 g/L glucose) plus supplementation with 10 mM iron (II) sulfate, 10 mM iron (III) sulfate, and 10 mM iron (II) gluconate dihydrate. Fungal transformants were incubated for 5 days at 30° C. at 150 rpm. The abundance of recombinant bovine myoglobin secreted into the feeding medium was measured as described in Example 1. As shown in FIG. 12-B, more than 40 μg/L of recombinant bovine myoglobin was detected in the feeding medium of cultures supplemented with iron (II) gluconate dehydrate. Supplementation with iron (II) sulfate and iron (III) sulfate did not improve myoglobin expression and secretion, averaging 20 μg/L of recombinant bovine myoglobin. Untransformed strain (C-) was used as a negative control.

### Example 5

pTrPdc-MyoTr (FIG. 6) and pTrGpd-MyoTr (FIG. 7) plasmids were assembled using pTrEno-MyoTr as parent vector. T. reesei promoters P_{pdc} and P_{gpd} were amplified from T. reesei genomic DNA using primers Ppdc_SbfI-F/Ppdc_PacI-R and Pgpd_SbfI-F/Pgpd_PacI-R, respectively. The promoter regions of these genes were acquired from the genome sequence of T. reesei (https://mycocosm.jgi.doe.gov/Trire2/Trire2.home.html). P_{pdc}, the promoter of pdc gene, is located from 106110 bp to 107414 bp in scaffold 8 (GenBank No. CP016233.1); P_{gpd}, the promoter of gpd gene, is located from 1561323 bp to 1562759 bp in scaffold 1 (GenBank No. CP020877.1). Pdc gene promoter (P_{pdc}, 1,305 bp upstream fragment starting from the start codon of pdc gene) and the gpd promoter (P_{gpd}, 1,437 bp upstream fragment starting from the start codon of the gpd gene) were amplified by PCR using the genomic DNA of T. reesei QM9414 as the template. All primers used are listed in Table 2: restriction sites are in bold. The vectors pTrPdc-MyoTr and pTrGpd-MyoTr were developed as follows: enolase promoter was released from pTrEno-MyoTr after digestion with restriction enzymes SbfI and PacI, resulting in two DNA fragments of 1.5 and 7.2 kb. The vector fragment (7.2 kb) was gel purified. The promoters amplified by PCR using primers Ppdc_SbfI-F/Ppdc_PacI-R and Pgpd_SbfI-F/Pgpd_PacI-R were cloned into pCR-TOPO (Invitrogen) and propagated in E. coli DH5a following the manufacturer's instructions. Sequences were analyzed in Macrogen, Korea. Plasmids containing the correct sequences were digested with SbfI and PacI and fragments containing the promoter regions were gel purified. Gel purified promoters and parent vector were ligated with T4 DNA ligase (NEB).

TABLE 2

| | Primer | Amplifi- | |
|---|---|---|---|
| Primer | Sequence | cation | |
| name | (5′-3′) | target | |
| Ppdc_Sbff- | GAGACCTGCAGGATGAAA | pdc | SEQ. ID |
| F | GGAGGGAGCATTCTTCGA | promoter | NO: 21 |
| | C | | |
| Ppdc_PacI- | CATTCTCATGTTGGTTAA | pdc | SEQ. ID |
| R | TTAAGATTGTGCTGTAGC | promoter | NO: 22 |
| | TGCGCT | | |
| Pgpd_SbfI- | GCTCCTGCAGGGACGCAG | pdc | SEQ. ID |
| F | AAGAAGGAAATCGCC | promoter | NO: 23 |
| Pgpd_PacI- | CATTCTCATGTTGGTTAA | pdc | SEQ. ID |
| R | TTAATTTGTATCTGCGAA | promoter | NO: 24 |
| | TTGAGCTTGCG | | |

The expression cassettes were released from pTrPdc-MyoTr and pTrGpd-MyoTr by restriction digestion with SbfI and XhoI (NEB), and transformation was performed as described in Example 1. After 3 days, the putative strains were transferred to selective PDA plates containing 100 μg/mL hygromycin. Seven and six putative transformants with each construct, pTrPdc-MyoTr (5.1A-5.1G) and pTrGpd-MyoTr (5.2A-5.2F), respectively, were grown in selective PDA plates for at least three generations.

Transformants harboring expression cassettes from plasmids pTrPdc-MyoTr (5.1A-5.1G) and pTrGpd-MyoTr (5.2A-5.2F) were grown in feeding medium (MA with 10 g/L glucose). The recombinant bovine myoglobin was secreted into the feeding medium and was detected in the feeding medium using ELISA. The relative abundance of recombinant bovine myoglobin was measured as described in Example 1. ELISA from the feeding media detected that 4 out of 7 transformants containing the cassette pTrPdc-MyoTr expressed and secreted more than 50 μg/L of recombinant bovine myoglobin, and 3 out of 6 transformants containing cassette pTrGpd-MyoTr expressed and secreted more than 60 μg/L of recombinant bovine myoglobin (shown in FIG. 13). Untransformed strain (C-) was used as a negative control.

### Example 6

Recombinant bovine myoglobin was collected by centrifugation from the feeding medium or culture supernatants. Then, the protein was concentrated and isolated using tangential flow filtration (TFF). Spores from strain 5.2F from Example 5 (containing pTrGpd-MyoTr cassette) were cultured in 2-L flasks containing 500 mL of MA with 10 g/L glucose and supplemented with 10 mM iron (II) gluconate dihydrate. Fungal transformants were incubated for 5 days at 30° C. at 150 rpm. The culture supernatants were cleared by centrifugation at 15,000×g for 1 h at 4° C. Clarified media (1 L per experiment) was processed using Minimate EVO Tangential Flow Filtration System (Pall Laboratories) and passed through a 10 kDa MWCO PES Minimate Capsule (Pall) at 5 mL/min with no back pressure applied. The flow-through (<10 kDa) was discarded and the clarified media circulated until it had reduced in volume to 5 mL. The sample was diafiltrated with 100 mL of lyophilization buffer (10 mM ammonium bicarbonate, pH 8.2) four times. The retentate (5 ml) was harvested and 10 mL of lyophilization buffer was used to wash the capsule membrane. The procedure was repeated twice to concentrate the total cleared culture medium volume (2 L). The 10 mL of retentate and 20 mL washed sample were combined and frozen with liquid nitrogen. The frozen extract was freeze-dried for 48 h.

The relative abundance of recombinant bovine myoglobin was measured as described in Example 1. Culture supernatants without any concentration step contained 79 μg/L of recombinant bovine myoglobin. After TFF step, 131 μg of recombinant bovine myoglobin was recovered, resulting in 82% recovery rate.

### Example 7

pTrPdcBle-ALAS (FIG. 8) is a plasmid containing the 5-aminolevulinic acid (ALAS) CDS from Trichoderma reesei and a Strepotalloteichus hindustanus bleomycin/phleomycin resistance gene (ble) as selectable marker (vector diagram shown in FIG. 14) This vector was designed for insertion at pdc gene in T. reesei chromosome and allows the co expression of ALAS in the T. reesei strain 5.2F from Example 5. The plasmid pTrPdcBle-ALAS was assembled using pTrPdc-MyoTr as parent vector. CDS from ble gene was obtained from pPICZalphaA (Invitrogen) and amplified by PCR using Gibson Assembly method. As shown in Table 3, primers for assembly contain 20 bp from ble gene and 20 bp from pgk promoter in pTrPdc-MyoTr (ble_Ppgk-F, sense), and 20 bp from cbh1 terminator (ble-Tcbh1-R, antisense); 20 bp from cbh1 terminator and 20 bp from ble gene in pPICZalphaA (Tcbh1-ble-F, sense), and 20 bp from pgk promoter and 20 bp from ble gene (Ppgk-ble-R, antisense). In Table 3, partial gene sequences from ble gene in primers are in bold and italics, whereas sequences from P_{pgk} and T_{cbh1} are underlined. PCR fragments were gel purified and assembled using Gibson Assembly Master Mix (NEB) following the manufacturer's protocol.

TABLE 3

| | Primer | Amplifi- | |
|---|---|---|---|
| Primer | Sequence | cation | |
| name | (5′-3′) | target | |
| ble_ | CCACGCCGATTTCCTGCCA | ble gene | SEQ ID |
| Ppgk-F | AAATGGCCAAGTTGACCAG | and | NO: 25 |
| | | pTrPdc- | |
| | | MyoTr | |
| | | plasmid | |
| ble- | CAGGCTTTCGCCACGGAGC | ble gene | SEQ ID |
| Tcbh1-R | TTCAGTCCTGCTCCTCGGC | | NO: 26 |
| | CAC | | |
| Tcbh1- | CCGAGGAGCAGGACTGAAG | pTrPdc | SEQ ID |
| ble-F | CTCCGTGGCGAAAGCCTG | plasmid | NO: 27 |
| | | from | |
| | | cbh1 | |
| | | terminator | |
| Ppgk- | GCACTGGTCAACTTGGCCAT | Plasmid | SEQ ID |
| ble-R | TTTGGCAGGAAATCGGCGTG | pTrPdc- | NO: 28 |
| | G | MyoTr | |
| | | from pgk | |
| | | promoter | |
| ALAS- | CAAATTAATTAACCAACATG | ALAS with | SEQ ID |
| PacI-F | AGAATGAGAATGGACTC | PacI | NO: 29 |
| ALAS- | GCCTCTAGATTAGGCGGAGA | ALAS with | SEQ ID |
| Xbal-R | CGTCCATG | XbaI | NO: 30 |
| ALAS-F | CCAACATGAGAATGGACTCCG | ALAS gene | SEQ ID |
| | | | NO: 31 |
| ALAS-R | TTAGGCGGAGACGTCCATGAC | ALAS gene | SEQ ID |
| | CC | | NO: 32 |

E. coli DH5α was transformed with the assembly reaction to obtain pTrPdcBle-MyoTr plasmid. The sequence from four resulting transformants' plasmid DNA was analyzed (Macrogen, Korea).

On the other hand, ALAS gene was amplified from T. reesei QM6a genome using primers containing a Kozak consensus sequence at the 5′ end (primers ALAS-F and ALAS-R, SEQ ID NO: 31 and 32, respectively). Then, the ALAS amplicon with the Kozak sequence was amplified using primers containing PacI and XbaI restriction sites (ALAS-PacI-F and ALAS-XbaI-R, SEQ ID NO: 29 and 30, respectively). All primers are listed in table 3; the restriction sites are in bold. ALAS gene amplified by PCR was cloned into pCR-TOPO (Invitrogen) and propagated in E. coli DH5α. The plasmid containing the correct sequence was digested with PacI and XbaI to release the ALAS gene flanked with PacI and XbaI restriction sites. Also, pTrPdcBle-MyoTr plasmid from assembly reaction was digested with PacI and XbaI. Then, the plasmid and the ALAS fragment gene were gel purified and ligated with T4 DNA ligase (NEB). The resulting vector pTrBle-ALAS was used for transformation in T. reesei QM6a pTrEno-MyoTr strain 5.2F (from Example 5).

T. reesei 5.2F was transformed with pTrPdcBle-ALAS by the protoplast method as described in Example 1. After 3 days, the putative strains were transferred into selective PDA plates containing 300 μg/mL phleomycin (Invivogen). Five putative transformants were recovered and cultured in MA medium with 10 g/L glucose and 25 nM pyridoxine hydrochloride (without iron supplementation) as described in Example 1. The putative transformants were purified on selective plates containing 50 μg/mL phleomycin for three generations. Genomic insertions in the five strains were confirmed by PCR using primers ALAS-F and ALAS-R.

ELISA from the feeding media showed that the five strains expressing ALAS (via pTrEno-MyoTr and pTrPdcBle-ALAS insertions) expressed and secreted more than 100 μg/L of recombinant bovine myoglobin (shown in FIG. 15-A). T. reesei transformant 7E expressed 150 μg/L of recombinant bovine myoglobin in average. Untransformed strain (C-) was used a negative control.

### Example 8

To assess the effects of iron supplementation in the T. reesei strain that co expressed bovine myoglobin and ALAS (strain 7E from Example 7), the strain 7E was cultured using heme-iron and non heme-iron supplementation.

Spores from 7E were cultured in 125-mL flasks containing 20 mL of MA medium with 10 g/L glucose, 25 nM pyridoxine hydrochloride, and supplemented with 20 mg/L of hemin (heme-iron), 10 mM iron (II) sulfate, 10 mM iron (III) sulfate, and 10 mM iron (II) gluconate dihydrate. Fungal strains were incubated for 5 days at 30° C. at 150 rpm.

ELISA from the feeding medium detected that the strain 7E expressed and secreted 190 μg/L of recombinant bovine myoglobin in average when supplemented with 10 mM iron (II) gluconate. The lowest yield was obtained in culture supplemented with 20 mg/L hemin, yielding an average of 95 μg/L of recombinant bovine myoglobin (shown in FIG. 14-B).

In the above, the present invention has been mainly described in its preferred embodiment. Those who have ordinary knowledge in the technical field to which the present invention belongs will understand that the present invention can be realized in a modified form within a range that does not deviate from the essential characteristics of the present invention. Therefore, the disclosed examples should be considered from a descriptive point of view rather than a limiting point of view.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others or ordinary skill in the art to understand the embodiments disclosed herein.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

Although this invention has been described with a certain degree of particularity, it is to be understood that the present disclosure has been made only by way of illustration and that numerous changes in the details of construction and arrangement of parts may be resorted to without departing from the spirit and the scope of the invention.

It should be understood that it is contemplated and within the scope of the present invention that any feature that is enumerated above can be combined with any other feature that is enumerated above as long as those features are not incompatible. The present invention comprises at least products (such as DNA sequences or amino acid sequences), compositions comprising the products, and methods of making and methods of using these products. In an embodiment, the present invention relates to methods of expressing and/or collecting myoglobin using the plasmids and ORFs of the sequences disclosed herein. In a variation, the present invention relates to using the filamentous fungi disclosed herein. In a variation, the present invention relates to optimizing the codons of the ORFs for the filamentous fungi disclosed herein, optionally performed using software. In a variation, the present invention relates to products and compositions comprising the DNA sequences disclosed herein.

What is claimed is:

1. A method to express and collect myoglobin using a fungus, the method comprising:
optimizing at least one codon in a gene that codes for myoglobin for a fungal host;
inserting the gene into a plasmid;
transforming the fungal host with the plasmid and,
in response to myoglobin being expressed and secreted extracellularly to a feeding media, collecting the myoglobin from the feeding media.

2. The method of claim 1, wherein the fungus is a mesophilic and filamentous fungus.

3. The method of claim 2, wherein the mesophilic and filamentous fungus comprises Trichoderma reesei.

4. The method of claim 1, wherein optimization of the at least one codon for the fungal host occurs via software.

5. The method of claim 1, wherein the plasmid comprises a pTrEno-derived plasmid with a strong constitutive promoter, such as the promoters glyceraldehyde-3-phosphate dehydrogenase (gpd) and pyruvate decarboxylase (pdc).

6. The method of claim 1, further comprising:
purifying the myoglobin.

7. A DNA sequence comprising a codon-optimized bovine myoglobin DNA sequence, wherein the DNA sequence comprises:
a PacI site;
an XbaI site;
a Kozak sequence;
DNA that encodes a signal peptide for peptide secretion; and
an open reading frame (ORF) encoding for myoglobin, wherein the Kozak sequence is disposed between the PacI site and the DNA that encodes the signal peptide, and wherein the ORF encoding for myoglobin is disposed between DNA that encodes the signal peptide and the XbaI site.

8. The DNA sequence of claim 7, wherein the ORF encoding for myoglobin comprises an ATG codon proximate the DNA that encodes the signal peptide and a stop codon proximate the XbaI site.

9. The codon-optimized bovine myoglobin DNA sequence of claim 7, wherein the ORF encoding for myoglobin is a Bos taurus codon-optimized to a Trichoderma reesei host.

10. The DNA sequence or claim 7, comprising SEQ ID NO 1.

11. The DNA sequence of claim 7, wherein a DNA sequence has at least 95% sequence homology to SEQ ID NO 1.

12. The method of claim 1, wherein the gene inserted into the plasmid comprises SEQ ID NO 1.

13. The method of claim 1, wherein a yield of myoglobin is at least 40 μg/L.

14. The method of claim 13, wherein the yield of myoglobin is at least 60 μg/L.

15. The method of claim 13, wherein the yield of myoglobin is at least 95 μg/L.

16. The method of claim 13, wherein the yield of myoglobin is at least 150 μg/L.

17. The method of claim 1, wherein the plasmid comprises a pTrEno-derived plasmid with a strong constitutive promoter, such as the promoters glyceraldehyde-3-phosphate dehydrogenase (gpd) and pyruvate decarboxylase (pdc).

18. The method of claim 6, wherein the myoglobin is co-expressed with one or more of ALAS, ALAD, PGDB, UROS, UROD, PPO, or FC.

19. Recombinant myoglobin made by the method of claim 1.

20. Recombinant myoglobin, collected from a feeding media wherein the recombinant myoglobin has been encoded by the DNA sequence of claim 7.
